(19) United States
(12) Patent Application Publication
(10) Pub. No.: US 2026/0085129 A1
(43) Pub. Date: Mar. 26, 2026

(54) CD163 BINDING PROTEIN

(71) Applicant: ECO ANIMAL HEALTH LTD., London (GB)

(72) Inventors: Christine TAIT-BURKARD, Roslin (GB); Charles Edward OWEN, Haywards Heath (GB); Hafid Abdelaali BENCHAOUI, London (GB)

(73) Assignee: ECO ANIMAL HEALTH LTD., London (GB)

(21) Appl. No.: 19/119,956

(22) PCT Filed: Oct. 11, 2023
(86) PCT No.: PCT/GB2023/052638

§ 371 Date: Apr. 10, 2025

(30) Foreign Application Priority Data
Oct. 11, 2022 (GB) 2214979.3

(51) Int. Cl. C07K 16/28; A61K 39/00; A61P 31/14

(52) U.S. Cl. CPC C07K 16/2896; A61P 31/14; A61K 2039/505; C07K 2317/569; C07K 2317/76; C07K 2317/92

(57) ABSTRACT

The present disclosure provides an antibody which binds to porcine CD163, wherein said antibody binds to the membrane-bound form of porcine CD163 on cells and does not bind significantly to the soluble form of porcine CD163. The present disclosure also provides a combination of said antibody with one or more further anti-porcine CD163 antibodies or binding proteins. Preferred combinations are those in which each antibody or binding protein binds to a different epitope in porcine CD163, and wherein said combination of anti-porcine CD163 antibodies or binding proteins are provided in a single construct. Nucleic acid molecules, expression vectors and compositions are also provided.

This application is a U.S. National Phase filing under 35 U.S.C. § 371 of International Application No. PCT/GB2023/052638, filed Oct. 11, 2023, and published as WO 2024/079463 A1 on Apr. 18, 2024, which claims benefit of priority from G.B Patent Application No. 2214979.3, filed Oct. 11, 2022, the entire contents of which applications are incorporated herein by reference.

The instant application contains an electronic sequence listing. The contents of the electronic sequence listing 69.129.159273-17 Sequence Listing XML 4599.039.xml; Size: 41,176 bytes; and Date of Creation: Apr. 3, 2025, is herein incorporated by reference in its entirety.

This invention relates generally to the field of binding proteins which bind to CD163 (Cluster of Differentiation 163), in particular antibodies, and in particular binding proteins and antibodies that bind to membrane-bound porcine CD163 whilst showing no significant binding to soluble forms of porcine CD163. Such anti-CD163 binding proteins and antibodies have therapeutic and protective uses, in particular when combined with other anti-CD163 binding proteins, such as in the treatment or prevention of infections such as Porcine Reproductive and Respiratory Syndrome (PRRS) virus (PRRSV) infections, for example reducing their incidence and severity. Binding protein and antibody-based compositions, methods and kits are also provided.

Porcine Reproductive and Respiratory Syndrome (PRRS) is one of the most devastating viral pig diseases worldwide and causes huge economic losses to the pig farming industry. The causative agent is PRRSV, an enveloped RNA virus classified in the family Arteriviridae within the order Nidovirales. PRRSV has a restricted host and cell tropism, with porcine alveolar macrophages (PAMs) as important target cells. Clinical symptoms are diverse, but include respiratory distress and respiratory disease in young pigs and piglets, late-term abortion and still-births in gilts and sows, foetal reabsorption in early pregnancy, reduced survivability if the piglets are born alive, and reduced growth in finishing pigs. Due to reduction or loss of pregnancies, death in young piglets, and decreased growth rates in all PRRSV infected pigs, it is estimated that more than $650 m is lost annually to pork producers in the US alone (Holtkamp et al., 2013, Journal of Swine Health and Production, 21(2) 72-84). A 2021 study (Renken et al., 2021, Porcine Health Management, January 4; 7(1):3) on German PRRSV endemic farms calculated median losses at €74, 181/farm (€255 per sow).

All currently known PRRSV isolates fall into one of two species, PRRSV-1 or PRRSV-2, which have only about 60% identity at the nucleotide level, although they both cause long-term infections and produce similar clinical signs. PRRSV-1 was first identified in Europe and tends to be found in European PRRSV isolates or strains, whereas PRRSV-2 was first identified in North America and tends to be found in Asian or American isolates or strains (see review by Stoian and Rowland, 2019, Vet. Sci., 6, 9).

Within each species, there is significant diversity with a large number of strains/subtypes identified, including new highly pathogenic strains that have emerged since 2006, particularly in China and Vietnam. Similar highly pathogenic strains have also emerged elsewhere, stretching from the Malaysian peninsula to southern Russia and these present a growing threat to the pig population (An et al., 2011, Emerging Infect Dis 17(9):1782). In China alone, more than 20 million pigs were culled because of PRRSV infection annually in 2006 and 2007 (An et al., 2010, Emerging Infect Dis 16(2):365). More recently, case reports of virulent strains causing outbreaks in Europe indicate the growing emergence of PRRSV as a threat (Sinn et al., 2016, Porcine Health Management (2):28).

The scavenger receptor CD163 is a key entry mediator for PRRSV infection and thus has a key role in PRRSV infection. CD163 is a 130 kDa type I transmembrane protein which has a signal peptide followed by nine scavenger-receptor cysteine rich (SRCR) domains, each approximately 100 amino acids in length, with a 35 amino-acid proline-serine-threonine (PST)-rich region (PST-1) separating SRCR domain 6 (SRCR6) and SRCR7. A second PST-rich region (PST-2) connects SRCR9 with the transmembrane domain and a short cytoplasmic tail, which contains a functional internalization motif. Surface expression of CD163 is restricted to cells of the monocyte-macrophage lineage. The SRCR5 domain of CD163 has been identified to play a significant role in the infection of porcine alveolar macrophages by PRRSV (Gorp et al., 2010, J. of Virology, March 3101-3105).

The precise mechanism of PRRSV entry is unknown. However, as part of this mechanism, PRRSV is thought to enter the endosomal compartment of cells, in which an interaction between CD163 and the GP2-GP3-GP4 heterotrimer of PRRSV mediates the uncoating of the virus and the release of the viral genome into the cytoplasm.

One proposed treatment option for PRRSV includes some kind of genetic knockout or gene editing of CD163, in order to make pigs resistant to PRRSV infection, and then breeding these pigs to propagate the genetic modification (Burkard et al., 2017, PLoS Pathogens 13(2):e1006206). Although this has been shown to work quite effectively, this treatment will be complex and time consuming in terms of being able to treat a significant proportion of the porcine population. In addition, and importantly, there is significant resistance in many markets to techniques involving the genetic modification of animals, for example, when it comes to the desirability of animal products produced from such animals.

The most common medical intervention used to limit the economic impact of PRRS is vaccination. Vaccines are used in all geographies where the disease is prevalent. However, they are only used under defined scenarios due to safety concerns. Two types of vaccines are used, either killed virus vaccines or (in the majority of cases) modified live vaccines (MLV). However, currently vaccines are only partially effective and add most value when deployed within an integrated approach to disease management where concurrent biosecurity and husbandry decisions are closely aligned. The reasons behind lack of vaccine efficacy are complex but the high genetic diversity of PRRSV populations coupled with the biology of the virus (tropism for alveolar macrophages and high mutability) are such that the best results are seen when the vaccine strain and the circulating strain are closely matched in terms of immunogenicity (reviewed by Nan et al., 2017. Front. Immunol. 8: 1635). Additionally, live vaccine strains can recombine with field strains to produce new field strains which may be pathogenic. Thus, MLVs can only be used in certain circumstances, which further limits their use.

There are currently no anti-viral treatment options for PRRSV infections.

More recently, alternative therapeutic or preventative options in the form of binding proteins and antibodies directed to porcine CD163, which can act to reduce or prevent PRRSV infection, have been developed.

There is however still a need for alternative and preferably improved treatment and prevention options for PRRSV infection (or other CD163 mediated infections) which can readily be used to treat or prevent infection in significant numbers of animals.

The present invention provides the means for such an alternative therapeutic or preventative option in the form of antibodies that have the ability to bind to the membrane-bound form of porcine CD163 on cells, but which advantageously do not bind significantly to soluble forms of porcine CD163. Such antibodies (or binding proteins comprising such antibodies) are sometimes referred to herein as membrane-specific CD163 antibodies (or binding proteins). Such antibodies (or binding proteins) have the ability to target only the membrane-bound form of CD163. In other words, they do not target, or do not significantly target, soluble CD163. This is advantageous as soluble CD163 is cleaved from the cell surface and can be found at high concentrations in the serum during some infections of pigs. Thus, soluble CD163 has the potential to effectively act as a sink for anti-CD163 antibodies that are used in therapy, and thereby interfere with their action. However, the antibodies of the present invention, due to their ability to distinguish or discriminate between the membrane (cell-surface) and soluble forms of CD163, should not be decoyed by such soluble (shed) antigen, but would instead target the cells expressing CD163 which are also the cells which are targeted by PRRSV during infection. Indeed, the antibodies of the invention have been shown to provide resistance to such soluble forms of CD163, e.g. have been shown to result in compositions that still function very effectively to inhibit PRRSV infection in the presence of soluble CD163.

In particular, the antibodies of the present invention have been shown to have excellent activity to inhibit PRRSV infection when combined with other CD163 antibodies. Without wishing to be bound by theory, it is believed that the antibodies of the present invention can help target the other CD163 antibodies to cells expressing CD163, thereby enabling infection by PRRSV to be reduced or blocked in a highly efficient way.

Extremely good results are observed when two anti-CD163 antibodies are paired together (e.g. an antibody of the invention with a second different anti-CD163 antibody). Such antibodies are sometimes referred to herein as bi-paratopic anti-CD163 antibodies. These bi-paratopic constructs show good inhibition of PRRSV-1 infection, although the effect is reduced in the presence of high concentrations of soluble CD163, and also some inhibition of PRRSV-2 infection. Even better results are observed when three anti-CD163 antibodies are put together (i.e. an antibody of the invention with a second and a third different anti-CD163 antibody). Such antibodies are sometimes referred to herein as tri-paratopic anti-CD163 antibodies. Advantageously, the tri-paratopic constructs of the invention can inhibit both PRRSV-1 and PRRSV-2 infection extremely effectively, even in the presence of high concentrations of soluble CD163, such as would likely be found in an infected pig as described elsewhere herein, for example, a pig suffering from a viral or bacterial infection, e.g. a non-PRRSV infection such as a Lawsonia intracellularis infection, or a pig with complex (multi-pathogen) disease or a pig with multiple (or mixed) infections, or a pig with a severe infection. Such pigs are often encountered in the field.

Thus, it is believed that the antibodies and constructs of the present invention (for example other binding proteins comprising a CD163 antigen binding domain as described herein) can provide a new type of therapeutic molecule that can preferentially target cell membrane forms of CD163 and hence provide very effective therapeutic options for the treatment of both PRRSV-1 and PRRSV-2 infections. As such antibodies do not bind significantly to soluble forms of CD163 they also have the potential to be effective at lower doses which is a yet further advantage both to the animal being treated and from a cost perspective.

In one embodiment, the present invention provides a binding protein, for example an antibody, which binds to CD163, for example porcine CD163, wherein said binding protein or antibody:
(i) binds to the membrane-bound form of CD163, for example porcine CD163, on cells; and
(ii) does not bind significantly to the soluble form of CD163, for example porcine CD163.

In one embodiment, the present invention provides a binding protein, for example an antibody, which binds to porcine CD163, wherein said binding protein or antibody:
(i) binds to the membrane-bound form of porcine CD163 on cells; and
(ii) does not bind significantly to the soluble form of porcine CD163.

As discussed elsewhere herein, preferred antibodies (or binding proteins) of the invention and suitable for use in the therapeutic methods described herein have the ability to bind to the SRCR5 domain of CD163 (e.g. porcine CD163), for example have an epitope (or part of an epitope) in the SRCR5 domain of CD163. In addition, preferred antibodies (or binding proteins) have the ability to inhibit PRRSV-2 infection.

In a further embodiment, the present invention provides a binding protein, for example an antibody, comprising at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTFSSYA (SEQ ID NO:2), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of IGWTGGTT (SEQ ID NO:3), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of AADQAGWRTAGVRNTYEYDY (SEQ ID NO:4), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTFSSYA (SEQ ID NO:2),
(ii) a variable heavy (VH) CDR2 that comprises the amino acid sequence of IGWTGGTT (SEQ ID NO:3), and
(iii) a variable heavy (VH) CDR3 that comprises the amino acid sequence of AADQAGWRTAGVRNTYEYDY (SEQ ID NO:4).

As described elsewhere herein, said antibodies (or binding proteins) preferably have the ability to bind to the membrane-bound form of CD163, e.g. porcine CD163, on cells, but do not bind significantly to the soluble form of CD163, e.g. porcine CD163.

Certain preferred embodiments of the invention provide an antibody (or binding protein) which binds to CD163, for example porcine CD163, comprising a VH domain that has or comprises the amino acid sequence of SEQ ID NO: 1, or a sequence substantially homologous thereto. In some embodiments, such antibodies (or binding proteins) also comprise a VL domain which comprises up to three light chain CDRs, and preferably three light chain CDRs.

In a preferred embodiment the present invention provides an antibody (or binding protein) which binds to CD163, for example porcine CD163, comprising a VH domain that has or comprises the amino acid sequence of SEQ ID NO: 1, or a sequence having at least 70%, 75% or 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, the present invention provides an antibody (or binding protein), which binds to CD163, for example porcine CD163, comprising a VH domain that has or comprises the amino acid sequence of SEQ ID NO: 1. In some embodiments, such antibodies (or binding proteins) also comprise a VL domain which comprises up to three light chain CDRs, and preferably three light chain CDRs.

As described elsewhere herein, said antibodies (or binding proteins) preferably have the ability to bind to the membrane-bound form of CD163, e.g. porcine CD163, on cells, but do not bind significantly to the soluble form of CD163, e.g. porcine CD163.

As described above, the present invention provides binding proteins, for example antibodies or binding proteins comprising an antigen binding domain, which bind to (or specifically recognise or specifically bind to) CD163, preferably porcine CD163. CD163 is also known as M130, MM130, SCAR1, Macrophage-associated antigen, Hemoglobulin scavenger receptor, or Scavenger receptor cysteine rich Type 1 protein M130.

CD163 is a 130 kDa type I transmembrane protein which has a signal peptide followed by nine scavenger-receptor cysteine rich (SRCR) domains, each approximately 100 amino acids in length, with a 35 amino-acid proline-serine-threonine (PST)-rich region (PST-1) separating SRCR domain 6 (SRCR6) and SRCR7. A second PST-rich region (PST-2) connects SRCR9 with the transmembrane domain and a short cytoplasmic tail, which contains a functional internalization motif. Surface expression of CD163 is restricted to cells of the monocyte-macrophage lineage.

The binding proteins or antibodies of the present invention thus bind to or are capable of binding to CD163. Binding proteins or antibodies of the invention are thus also sometimes referred to herein as anti-CD163 binding proteins or antibodies. In accordance with the present invention, the CD163 may be from any species, e.g. any mammalian species such as pig (porcine), human, cattle (bovine), dog (canine), cat (feline), sheep (ovine), horse (equine), mouse and monkey. In a preferred embodiment the CD163 is porcine CD163 and the antibodies (or binding proteins) bind to or are capable of binding to (or specifically recognise or specifically bind to) porcine CD163, e.g. can be referred to as anti-porcine CD163 antibodies.

Of particular relevance to the present invention, CD163 is expressed on the surface of porcine alveolar macrophages (PAMs), and is believed to play a vital role in the ability of various pathogens, including viral pathogens, notably PRRSV, to cause disease in pigs.

As mentioned above, the present invention also provides antibodies or binding proteins, preferably isolated antibodies or binding proteins, which bind to CD163 (preferably porcine CD163), wherein said antibodies (or binding proteins) bind to the membrane-bound form of CD163 on cells and do not bind significantly to the soluble form of CD163. Such antibodies (or binding proteins) of the invention are also sometimes referred to herein as membrane-specific antibodies (or binding proteins).

The term “membrane-bound form of CD163” or “membrane-bound form of CD163 on cells”, or other equivalent terms refers to CD163 which is attached to, associated with, embedded in, or otherwise bound to a cell membrane on a cell, or is a component of a cell membrane on a cell. Thus, the membrane-bound form of CD163 can be referred to, for example, as a cell surface form of CD163 or a cell surface CD163 molecule or a cell surface associated CD163 molecule, or a cell expressed CD163 molecule, or full length CD163 (e.g. including the transmembrane domain and optionally the cytoplasmic tail). Such membrane bound forms will thus in many cases represent (or correspond to) a native or natural form of CD163, for example the form found on cells which naturally express or overexpress CD163.

Unless otherwise evident, in the context of the present invention the term “cells” is used to refer to CD163-positive (CD163 expressing) cells. In the context of the present invention the term “cells” is used to refer to nucleus-containing cells.

Appropriate cell types which naturally express CD163 will be well known to a person skilled in the art and include monocytes and macrophages. A preferred cell type is porcine alveolar macrophages (PAMs). Alternatively. CD163 can be expressed or overexpressed in a membrane-bound form, e.g. by recombinant means (or by other means of engineering) in a cell type which does not normally express CD163, in other words a cell expressing a recombinant membrane-bound form of CD163, e.g. full length CD163, can be used.

Thus, in some embodiments, the membrane-bound form of CD163 on cells is the membrane-bound form of CD163 on PAMs and the antibodies (or binding proteins) of the invention have the ability to bind to PAMs, sometimes also referred to herein as porcine PAMs (or pPAMs). Put another way, in some embodiments, the membrane-bound form of CD163 on cells is PAM-associated CD163. In some embodiments, the membrane-bound form of CD163 on cells is the membrane-bound form of CD163 on cells that have been transfected with (and thus express or overexpress) CD163, e.g. a recombinant form of CD163, preferably porcine CD163. Thus, antibodies (or binding proteins) which bind to recombinant cells expressing CD163 are also included. Appropriate cells for transfection would be well known and described in the art and some examples are given elsewhere herein for example HEK293 and CHO cells. Typically CD163 negative cells are used, i.e. cells that do not express CD163 before they are transfected.

A preferred membrane-bound form of CD163 on cells is the 130 kDa native membrane-bound form, or a corresponding recombinant form. Such membrane-bound forms of CD163 comprise all regions of CD163 apart from the signal sequence (which is removed during intracellular processing and subsequent cell surface expression), e.g. comprise all nine SRCR domains plus both PST-rich regions, the transmembrane region and the cytoplasmic tail.

A preferred membrane-bound form of CD163 on cells comprises (or consists of) amino acid residues 47 to 1044 of SEQ ID NO:42 (porcine CD163). Thus, antibodies of the present invention preferably bind to this membrane-bound form of CD163 on cells. Antibodies of the invention may bind to a membrane-bound form of CD163 on cells that corresponds to this membrane-bound form of CD163 on cells (e.g. in a different CD163 isoform or CD163 from a different species).

Antibodies of the present invention preferably bind to the SRCR5 domain of CD163, e.g. porcine CD163 (or to an epitope comprising one or more residues in said SRCR5 domain). Thus, preferred membrane specific anti-CD163 binding proteins or antibodies of the present invention have the ability to bind to the SRCR5 domain or an epitope (or part of an epitope) in the SRCR5 domain, preferably the porcine SRCR5 domain, of CD163.

Thus, in some embodiments, the membrane-specific anti-CD163 antibodies of the invention do not bind to (or do not significantly bind to) CD163 molecules which comprise a deletion of or within, or a mutation within, the SRCR5 domain. Thus, in some embodiments, the membrane-specific antibodies of the invention do not bind to (or do not significantly bind to) the PST-2 domain or an epitope (or part of an epitope) in the PST-2 domain of CD163, e.g. porcine CD163.

Porcine forms of CD163 are preferably used to assess the binding capabilities of antibodies of the present invention, although equivalent forms from other species, e.g. other mammalian species, may also be used, for example to assess for cross-reactivity.

The sequences of CD163 in various species are well known and described in the art and can be obtained for example from various sequence databases, e.g. Uniprot. For ease of reference, the porcine CD163 has the Uniprot number Q2VL90 and is reproduced below for reference.

The sequence of the porcine SRCR5 domain is shown below and corresponds to residues 477-577 of Uniprot Q2VL90:

(SEQ ID NO: 41)
PRLVGGDIPCSGRVEVQHGDTWGTVCDSDFSLEAASVLCRELQCGTVVS
LLGGAHFGEGSGQIWAEEFQCEGHESHLSLCPVAPRPDGTCSHSRDVGW
VCS.

The sequence of porcine CD163 is shown below and corresponds to the full sequence of Uniprot Q2VL90:

(SEQ ID NO: 42)
MDKLRMVLHENSGSADFRRCSAHLSSFTFAVVAVLSACLVTSSLGGKDKELRLTGGENKC
SGRVEVKVQEEWGTVCNNGWDMDVVSVVCRQLGCPTAIKATGWANFSAGSGRIWMDHV
SCRGNESALWDCKHDGWGKHNCTHQQDAGVTCSDGSDLEMGLVNGGNRCLGRIEVKFQ
GRWGTVCDDNFNINHASVVCKQLECGSAVSFSGSANFGEGSGPIWFDDLVQNGNESALW
NCKHEGWGKHNCDHAEDAGVICLNGADLKLRVVDGVTECSGRLEVKFQGEWGTICDDGW
DSDDAAVACKQLGCPTAVTAIGRVNASEGTGHIWLDSVSCHGHESALWQCRHHEWGKHY
CNHDEDAGVTCSDGSDLELRLKGGGSHCAGTVEVEIQKLVGKVCDRSWGLKEADVVCRQ
LGCGSALKTSYQVYSKTKATNTWLFVSSCNGNETSLWDCKNWQWGGLSCDHYDEAKITC
SAHRKPRLVGGDIPCSGRVEVQHGDTWGTVCDSDFSLEAASVLCRELQCGTVVSLLGGAH
FGEGSGQIWAEEFQCEGHESHLSLCPVAPRPDGTCSHSRDVGVVCSRYTQIRLVNGKTPC
EGRVELNILGSWGSLCNSHWDMEDAHVLCQQLKCGVALSIPGGAPFGKGSEQVWRHMFH
CTGTEKHMGDCSVTALGASLCSSGQVASVICSGNQSQTLSPCNSSSSDPSSSIISEENGVA
CIGSGQLRLVDGGGRCAGRVEVYHEGSWGTICDDSWDLNDAHVVCKQLSCGWAINATGS
AHFGEGTGPIWLDEINCNGKESHIWQCHSHGWGRHNCRHKEDAGVICSEFMSLRLISENS
RETCAGRLEVFYNGAWGSVGRNSMSPATVGVVCRQLGCADRGDISPASSDKTVSRHMW
VDNVQCPKGPDTLWQCPSSPWKKRLASPSEETWITCANKIRLQEGNTNCSGRVEIWYGGS
WGTVCDDSWDLEDAQVVCRQLGCGSALEAGKEAAFGQGTGPIWLNEVKCKGNETSLWD
CPARSWGHSDCGHKEDAAVTCSEIAKSRESLHATGRSSFVALAIFGVILLACLIAFLIWTQKR
RQRQRLSVFSGGENSVHQIQYREMNSCLKADETDMLNPSGDHSEVQ.

The ability of an antibody (or binding protein) to bind to a membrane-bound form of CD163 on cells (or to cell surface expressed CD163) can be readily tested using methods that are well known and routine in the art, and any appropriate method can be used. For example, flow cytometry (e.g. FACS) can be used. In an exemplary flow cytometry method. CD163 expressing cells (e.g. PAM cells, e.g. pPAM cells, or cells expressing recombinant forms of CD163, e.g. cells transfected with CD163, e.g. full length CD163, e.g. transfected with a construct containing all regions of CD163, e.g. the signal sequence, all nine SRCR domains plus both PST-rich regions, the transmembrane region and the cytoplasmic tail) are incubated with the antibody (or binding protein) under investigation and the antibody (or binding protein) bound to the CD163 on the cell is detected by fluorescence, for example the antibody is fluorescently labelled. Such labelling can for example be carried out by incubating the cell-antibody mixture with a secondary antibody which recognises the antibody under investigation (e.g. an anti-myc antibody if the antibody under investigation is myc tagged) and a yet further antibody (third) antibody which is fluorescently labelled (such a third antibody recognises the second antibody). Alternatively, the second antibody can also carry the fluorescent label. Accordingly, if the antibody (or binding protein) under investigation binds to the membrane-bound form of CD163 on the cell, the cell becomes fluorescently labelled and such cells, and thus antibodies (or binding proteins) which have the ability to bind to a membrane-bound form of CD163 on cells, can be readily identified using a flow cytometer. A particularly preferred flow cytometry assay for testing for the ability of an antibody (or binding protein) to bind to a membrane-bound form of CD163 on cells is described in the Examples.

In some embodiments, PAM cells with a deleted SRCR5 domain can be used for these binding assays (e.g. as described in Burkard et al., 2017, PLoS Pathogens, 13(2):e1006206) in order to assess whether the antibody (or binding protein) has the ability to bind to the SRCR5 domain.

Another method for testing for the ability of an antibody (or binding protein) to bind to a membrane-bound form of CD163 is immunohistochemistry. Another method for testing for the ability of an antibody (or binding protein) to bind to a membrane-bound form of CD163 is microscopy (e.g. confocal microscopy) of cells that have become fluorescently labelled as a result of antibody (or binding protein) binding to membrane-bound CD163.

As discussed elsewhere herein, antibodies (or binding proteins) of the present invention do not bind significantly to (or do not bind to) the soluble form of CD163, e.g. porcine CD163.

The soluble form of CD163 refers to a form of CD163 which is present in solution or in a soluble phase. Thus, this form of CD163 is not associated with a membrane and is not particulate and not in the form of an insoluble aggregate or precipitate. A preferred form of soluble CD163 is (or corresponds to) CD163 which was associated with the surface of a cell, e.g. a PAM, or another macrophage or monocyte, and has been shed or lost from the cell membrane to become a soluble-form of CD163, for example by cleavage such as proteolytic cleavage (such a form of CD163 can also be referred to as “shed” or the “shed” form of CD163). Thus, a soluble form of CD163 can be a form which is derivable from the membrane bound form by cleavage, for example by native cleavage which occurs within the PST2 domain (likely site of cleavage between the residues HATG (residue 1041) and RSS).

A preferred soluble form of CD163 to which antibodies, etc., of the invention do not bind significantly is a soluble form of the membrane-bound CD163 that has been shed from (cleaved-off from) the cells. Thus, a soluble form of CD163 may comprise (or consist of) the same primary amino acid sequence as (or contained in) the membrane-bound form of CD163 on cells, or comprise (or consist of) a substantial portion (fragment) of the primary amino acid sequence of the membrane-bound CD163 on cells. For example, a soluble form of CD163 may comprise (or consist of) an amino acid sequence having at least 100, at least 200, at least 300, at least 400, at least 450, at least 500, at least 600, at least 700, at least 800, at least 900, or at least 950 amino acids that correspond to the amino acid sequence of the membrane-bound form of CD163 on cells. Such forms generally comprise or consist of fragments from the extracellular domain of CD163, e.g. fragments comprising or consisting of amino acid sequences from, in order from N-terminus to C-terminus in the CD163 molecule, SRCR1-6, PST-1, SRCR7-9 and PST-2, or fragments taken from amino acid residues 51 to 1044 or 51 to 1041 of SEQ ID NO:42. In some embodiments, a soluble form of CD163 has a sequence corresponding thereto (e.g. in a different CD163 isoform or CD163 from a different species).

In preferred embodiments of the present invention, the soluble CD163 maintains the tertiary structures which form naturally under physiological conditions, such as those physiological conditions present within a mammal, e.g. a pig. Thus, a soluble form of CD163 can be one in which the native tertiary structures are present, i.e. for example the soluble CD163 is not denatured. Accordingly, the soluble form of CD163 may be a non-denatured protein that retains a two-dimensional and/or three dimensional structure. The two-dimensional and/or three-dimensional structure of the soluble CD 163 may be a folded structure. Appropriate soluble forms can also be prepared recombinantly, e.g. can be recombinant or synthetic molecules.

A preferred soluble form of CD163 (e.g. a recombinant soluble form) comprises (or consists of) CD163-SRCR5-6, which corresponds to amino acid residues 477-682 of SEQ ID NO: 42. In some embodiments, a soluble form of CD163 has a sequence corresponding thereto (e.g. in a different CD163 isoform or CD163 from a different species).

Another preferred soluble form of CD163 (e.g. a recombinant soluble form) comprises (or consists of) CD163-SRCR4-7, which corresponds to amino acid residues 372-818 of SEQ ID NO:42 and includes the PST-1 domain. In some embodiments, a soluble form of CD163 has a sequence corresponding thereto (e.g. in a different CD163 isoform or CD163 from a different species).

Another preferred soluble form of CD163 (e.g. a recombinant soluble form) comprises (or consists of) CD163-SRCR1-9, which corresponds to amino acid residues 51-1028 of SEQ ID NO:42. In some embodiments, a soluble form of CD163 has a sequence corresponding thereto (e.g. in a different CD163 isoform or CD163 from a different species).

Another preferred soluble form of CD163 (e.g. a recombinant soluble form) comprises (or consists of) CD163-SRCR1-PST2, which corresponds to amino acid residues 51-1044 or 51-1041 of SEQ ID NO:42. In some embodiments, a soluble form of CD163 has a sequence corresponding thereto (e.g. in a different CD163 isoform or CD163 from a different species).

Soluble (or shed) CD163 may be found in the blood of subjects, e.g. pigs (serum CD163), but also may be found in the interstitial space of tissues. The soluble form of CD163 can thus exist naturally, or correspond to a naturally occurring form or native form of soluble CD163.

The soluble CD163 can be from any appropriate source, i.e. any sample or source in which the CD163 is present in a soluble form.

One appropriate and preferred source is recombinant CD163 (e.g. recombinant porcine CD163). Relevant forms of soluble recombinant CD163, e.g. porcine CD163, can readily be produced or synthesised using standard techniques, and for example the sequence information provided herein and elsewhere in the art. For example, recombinant CD163 constructs encoding desired soluble forms of CD163, such as those described elsewhere herein, can readily be constructed and expressed in appropriate cell lines, thereby allowing soluble CD163 protein to be isolated/purified.

In the case of CD163 shed from cells into the circulation, then an appropriate source might be blood or serum from a relevant subject (e.g. pigs or porcine subjects). For example, relatively low levels of soluble CD163 (e.g. around or up to 0.5 mg/ml) can be found in the serum of healthy pigs. In addition, higher levels of soluble CD163 (e.g. around or up to 4.5 mg/ml) can be found in the serum of infected pigs (pigs suffering from an infection) or pigs otherwise having an inflammatory response. Such higher levels of soluble CD163 can be found in pigs with any infection, e.g. any bacterial or viral infection, e.g. a non-PRRSV infection, which for example induces an inflammatory response or inflammation in the animals. However, a typical and exemplary infection which occurs frequently in the field is infection with Lawsonia intracellularis, and also infections with M hyo, P. multocida, and S. suis. It is also common in the field to have multiple infections occur concurrently (complex/multi pathogen disease) and such subjects (e.g. pigs or porcine subjects), or pigs with more severe infections, are also likely to have higher levels of soluble CD163 than those found in healthy subjects (e.g. pigs or porcine subjects). Thus, whole blood or serum from such healthy or infected pigs (e.g. serum derived from a pig infected with Lawsonia intracellularis or suffering from other infections) could provide a source of soluble CD163.

Soluble CD163 (or soluble CD163 related peptides) to which antibodies (or binding proteins) of the invention preferably do not bind significantly may thus be CD163 in a physiological fluid (e.g. serum), for example a physiological fluid (e.g. serum) from a healthy pig or an infected pig.

Exemplary soluble forms of CD163, e.g. recombinant CD163, as described above and elsewhere herein can be used in order to assess the ability of the binding proteins and antibodies to bind to soluble CD163. Exemplary are constructs containing appropriate extracellular portions of CD163, e.g. subsets of different CD163 SRCR domains such as CD163-SRCR1-PST2, CD163-SRCR1-9, CD163-SRCR4-7 or CD163-SRCR5-6. Equally other combinations of CD163 SRCR domains and fragments containing subsets of different CD163 SRCR domains, PST-1 and PST-2 domains can be used providing only extracellular parts are present. Porcine forms are preferably used to assess the antibodies of the present invention, although equivalent forms from other species, e.g. other mammalian species, may also be used, for example to assess for cross-reactivity.

Methods of assessing binding to (or ability to bind to) appropriate soluble forms of CD163 would be well-known to a person skilled in the art, and any appropriate method can be used.

A convenient and appropriate method for assessing binding includes in vitro binding assays such as ELISA assays to assess binding of antibodies or binding proteins to immobilised antigen, such as immobilised forms of soluble CD163 as described herein. The skilled person will be familiar with ELISA assays and readily able to establish suitable conditions to assess the ability of a binding protein or antibody to bind to CD163 in such an assay. A particularly preferred ELISA assay is described in the Examples section.

Preferred antibodies (or binding proteins) of the invention do not bind, or do not significantly bind, or do not measurably bind, to the soluble form of CD163, preferably porcine CD163, as assessed by ELISA. Exemplary soluble forms are as described elsewhere herein. Preferred soluble forms are pCD163 SRCR1-PST2 and pCD163-SRCR1-9, see for example as described in the Examples section.

A preferred method for assessing binding to (or ability to bind to) appropriate soluble forms of CD163 (e.g. porcine CD163) is a Surface Plasmon Resonance (SPR) assay (e.g. a BIACore assay). Suitable SPR assays are known in the art and are preferred because they allow binding to be more easily and consistently quantified. In certain preferred SPR assays, an appropriate form of soluble CD163 is captured (or immobilised) on a solid support (e.g. a sensor chip), for example via amine coupling (e.g. 2000 to 2500 or 2500 to 3500 Response Units (RU) CD163 is immobilized) and various concentrations (e.g. a dilution series, e.g. a doubling or trebling dilution series) of the binding proteins or antibodies to be tested is then injected. Preferred concentrations and flow-rates for injection are described in the Examples section. A preferred pH for assessment is pH 7.4. Exemplary soluble forms of CD163 for use in such SPR assays are as described elsewhere herein. Preferred soluble forms are CD163-SRCR1-PST2 or CD163-SRCR1-9 or CD163-SRCR4-7.

Such SPR assay methods can also conveniently be used to measure the binding kinetics of the antibody-antigen interaction, e.g. to determine kinetic parameters such as association rate (ka), dissociation rate (kd) and affinity (KD). In certain embodiments, measurements may be performed at 25° C. in a suitable buffer, e.g. a standard HEPES-EDTA buffer such as HBS-EP (sold by GE Healthcare Life Sciences, 0.01M HEPES PH 7.4, 0.15M NaCl, 3 mM EDTA, 0.0005% surfactant P20), at pH7.4. Kinetic parameters may be determined or calculated by any suitable model or software, for example by fitting the sensogram experimental data assuming a 1:1 interaction, for example using the BIAevaluation software. A particularly preferred SPR assay is described in the Examples section herein.

Thus, in a particularly preferred embodiment, binding proteins or antibodies of the present invention are deemed to not bind to the soluble form, or to not bind significantly to the soluble form of CD163 (e.g. porcine CD163) in (as determined in, when assessed in) a Surface Plasmon Resonance (SPR) assay (e.g. a BIACore assay).

In certain preferred embodiments, antibodies of the present invention that do not bind, or do not significantly bind to soluble CD163, e.g. when in VHH or VH format, have a non-detectable or essentially non-detectable binding affinity for soluble CD163 (e.g. porcine CD163), e.g. have a K_{D} (equilibrium dissociation constant affinity) in the range of 100 μM or higher (worse/less strong binding).

Thus, preferably, antibodies of the invention, e.g. when in VHH or VH format, have a binding affinity for soluble CD163 (e.g. soluble porcine CD163) that corresponds to a Ko of greater than 20 μM, 50 μM, 100 μM, 150 μM, 200 μM or 250 μM in (as determined in, when assessed in) a Surface Plasmon Resonance (SPR) assay (e.g. a BIACore assay), or is otherwise non-detectable by SPR or essentially so low that it is unmeasurable or the measurement is unreliable, e.g. cannot be fitted or properly fitted assuming a 1:1 interaction. Particular exemplary binding affinities are disclosed in the Examples. Thus, for example the H17B11 VHH antibody of the invention (as shown in Table A) has a binding affinity of at least 100 μM, e.g. at least 200 or 250 μM, as assessed in a Surface Plasmon Resonance (SPR) assay/BIACore assay.

Exemplary forms of soluble CD163 that can be used to assess such binding affinity are forms of recombinant soluble CD163, e.g. porcine CD163, as described herein, e.g. containing SRCR4-7 or SRCR1-9, e.g. recombinant porcine CD163 containing SRCR4-7 or SRCR1-9. Appropriate exemplary forms are described in the Examples section, for example the constructs pCD163-SRCR4-7 (optionally with a human (hu) Fc) or pCD163-SRCR1-9 (optionally with a huFc) or pCD163-SRCR1-PST2 (optionally with a His tag), preferably pCD163-SRCR1-PST2 or pCD163-SRCR1-9 or pCD163-SRCR4-7 (optionally with a huFc). pCD163-SRCR-FL-PST2, pCD163-SRCR-1-PST2 and pCD163-1-PST2 are used interchangeably to mean a porcine CD163 construct comprising the full length CD163 sequence from SRCR1 to PST2. Thus, the binding affinities above may be observed when or if the antibodies of the invention, e.g. in a VHH or VH format, are assayed using these constructs, e.g. in an SPR assay.

Importantly, antibodies (or binding proteins) of the present invention bind to the membrane-bound form of CD163 on cells and do not bind significantly to the soluble form of CD163. This combination of properties is potentially particularly important from the point of view of antibody-based therapies (e.g. treatment or prevention of PRRSV infection). As described above, without wishing to be bound by theory, antibodies which bind the membrane bound form of CD163 on cells (e.g. PAMs, porcine PAMs) but not the soluble form would not be decoyed by soluble (shed) CD163 in the circulation or in the interstitial spaces of subjects being treated but would instead target straight to the cell membranes of appropriate disease related cells (e.g. CD163 expressing PAMs which are targeted by PRRSV).

Preferred antibodies of the invention retain the ability to bind to the membrane-bound form of CD163 on cells and to not bind significantly to the soluble form of CD163 in the presence of any physiological concentration (e.g. any concentrations observed in the human or animal body) of soluble CD163. For example, the ability is retained even when high concentrations of soluble CD163 are present, for example concentrations of, or up to, or at least 0.4, 0.5, 1.0, 1.5, 2.0, 2.5, 3.0, 3.5, 4.0, 4.5, 5.0, 5.5 or 6.0 mg/l.

Preferred antibodies of the present invention thus discriminate between the membrane bound form of CD163 on cells and soluble forms of CD163. Thus, antibodies of the invention can positively discriminate for the membrane bound form of CD163 on cells. Antibodies of the present invention may thus be considered specific for the membrane-bound form of CD163 on cells.

Antibodies of the present invention may thus bind to a conformational epitope on CD163. Such a conformational epitope of CD163 is present on membrane-bound CD163 on cells but not present (or is significantly diminished or altered) on the soluble form of CD163. Without wishing to be bound by theory, it is believed that the conformational epitope on membrane-bound CD163 on cells to which antibodies of the present invention (e.g. H17B11) may bind may be generated by being tethered to the membrane via the PST2 domain, which epitope is then lost or changed once the CD163 molecule is shed or released. However, preferred antibodies of the invention do not bind to the PST2 region (e.g. do not bind directly to the PST2 region itself).

Thus, although corresponding (or identical) primary amino acid sequences (linear amino acid sequences) may be present in the membrane-bound form of CD163 on cells and in the soluble form of CD163, preferred antibodies of the present invention are advantageously able to discriminate between the different forms, e.g. by recognising a conformational rather than a linear epitope, e.g. recognise a neo-epitope or neo-conformational epitope present in the membrane-bound form of CD163 on cells but not in the soluble form of CD163.

Appropriate methods for arriving at the membrane specific anti-CD163 antibodies of the invention are provided in the Examples. However, a preferred protocol would involve immunization of an appropriate animal (e.g. a camelid, e.g. a llama) with membrane-expressed CD163, e.g. porcine CD163, e.g. in the form of cells (e.g. HEK or CHO cells) which recombinantly express CD163, for example recombinantly express a construct containing a significant proportion of the extracellular domain of CD163, e.g. a construct such as porcine (p)CD163-SRCR1-PST2 or pCD163-SRCR1-9, and/or with PAMs (or other cells with native expression of CD163). Antibody clones prepared from such immunizations could then be screened for membrane specific anti-CD163 antibodies or subjected to appropriate rounds of selection.

Preferred rounds of selection might involve the preparation of libraries of antibody clones, typically a phage display library, e.g. from the blood (PBMCs) of immunized animals, and then subjecting these clones to one or more appropriate rounds of selection. A preferred method of selection as used herein involved selections against both native membrane-expressed CD163 (here isolated pPAM cells) and recombinant cell-expressed CD163 (here HEK cells expressing pCD163-SRCR1-PST2). One or more counter-selections (or negative selections) can also be used, for example in the methods herein, counter-selections with both empty HEK wild-type cells and pPAMΔ5 cells (cells with deletion of SRCR domain 5) were used. Counter-selection in the presence of an excess of soluble CD163 would also be an appropriate additional or alternative selection step in order to obtain membrane-specific clones.

Once selection rounds have been carried out then screening for membrane specific anti-CD163 antibodies can take place.

A convenient and preferred way to do this screening would be to carry out flow cytometry (FACS) analysis using CD163 expressing cells which are known to be positive for the membrane bound form of CD163 (e.g. PAMs, or cells expressing a recombinant membrane bound form of CD163, e.g. as described elsewhere herein, e.g. a full length membrane-bound form of CD163). Preferably cells with a native form of CD163, e.g. PAMs are used. Candidate antibodies that have the ability to bind to the cell-expressed (membrane-bound) form of CD163 could then be identified.

The positive clones can then be subjected to further screening to assess whether or not they also do not have the ability to bind (or significantly bind) to soluble CD163, e.g. have the ability to discriminate between the membrane bound form of CD163 on cells and the soluble form. Again any appropriate method, e.g. BiaCore or ELISA can be used, although final confirmation is preferably done via SPR (e.g. BiaCore or an equivalent method) for accuracy and reliability. Appropriate soluble forms of CD163, e.g. recombinant soluble forms, are described elsewhere herein, and include pCD163-SRCR1-PST2 or pCD163-SRCR1-9 or pCD163-SRCR4-7.

Non-significant (insignificant) binding to the soluble form of CD163 generally means reproducibly (i.e. consistently observed) low or negligible binding to these forms of CD163. In some cases, insignificant binding can be considered to be at a background level, e.g. comparative to or not significantly different from a level observed in a negative control experiment, or at a non-detectable or very low affinity in (as determined in) for example an SPR assay. Appropriate tests for determining whether or not an antibody (or binding protein) does not bind or does not bind significantly to the soluble form of CD163 are described elsewhere herein.

In some embodiments, antibodies of the invention do not bind (e.g. do not measurably bind) to the soluble form of CD163.

Another convenient way of identifying (carrying out the screening for) antibodies which can bind to the membrane-bound form of CD163 on cells but not bind (or not significantly bind) to the soluble form of CD163 will be the use of some kind of competition assay, e.g. as part of a flow cytometry (FACS) analysis. Thus, an assay can be used where a sample of soluble CD163 (e.g. recombinant forms of soluble CD163 as described elsewhere herein) is introduced in order to assess whether the soluble CD163 has the ability to compete for the binding of a candidate antibody to a source of the membrane bound form of CD163 on cells. If the soluble CD163 can compete to a significant extent then this is indicative that the antibody candidate is not specific for the membrane bound form of CD163 on cells (as it also binds the soluble form). If the soluble CD163 cannot compete to a significant extent then this is indicative that the antibody candidate has the ability to discriminate between the membrane bound form of CD163 on cells and the soluble form.

In such a FACS assay a significantly reduced signal when the soluble form of CD163 is added indicates that the candidate antibody binds to both the membrane bound form on cells and soluble forms, i.e. does not discriminate, whereas a largely or significantly maintained signal when the soluble form is added indicates that the candidate antibody does not bind (or does not significantly bind) the soluble form but does bind the membrane bound form (or there would be no positive signal), i.e. that the antibody can distinguish between the membrane bound form of CD163 on cells and the soluble form of CD163.

Binding proteins of the invention can bind (e.g. specifically bind) to CD163, preferably to porcine CD163.

Preferred binding proteins of the invention are or comprise antibodies, and in particular VHH antibodies or single domain antibodies. However, embodiments as described herein which relate to antibodies, e.g. VHH antibodies or single domain antibodies, apply equally, mutatis mutandis, to other types of binding proteins, or vice versa.

Preferred binding proteins are any single polypeptide chains which can bind (e.g. specifically bind) to CD163, preferably to porcine CD163.

Appropriate types of binding protein which could be used in the invention are known in the art. For example, in some embodiments immunoglobulin based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold.

Thus, binding proteins that comprise an antigen binding domain are also preferred, in particular when that antigen binding domain is or comprises or is derived from an antibody (e.g. comprises the CDR and optionally FR regions of an antibody).

Binding proteins of the invention can preferably comprise multiple antibodies or antigen binding domains that bind to CD163, e.g. two or three different antibodies as discussed elsewhere herein.

As explained elsewhere herein, the antibodies of the present invention, due to their ability to distinguish between the membrane (cell-surface) and soluble forms of CD163, have the advantage that they should not be decoyed, or not significantly decoyed, or be subject to significantly less decoy, by soluble (shed) CD163 antigen, but would instead target the cells expressing CD163 which are also the cells which are targeted by PRRSV during infection. Indeed, the antibodies of the invention have been shown to provide resistance to such soluble forms of CD163, e.g. have been shown to result in compositions that still function very effectively to inhibit PRRSV infection in the presence of soluble CD163.

As well as advantageously having the property of membrane-specific binding, the antibodies of the present invention have also been shown to have the ability to inhibit PRRSV-2 infection.

In addition, the antibodies of the present invention have been shown to have excellent activity to inhibit PRRSV infection when combined with other CD163 antibodies. Without wishing to be bound by theory, it is believed that the antibodies of the present invention can help target the other CD163 antibodies to cells expressing CD163, thereby enabling infection by PRRSV to be reduced or blocked in a highly efficient way.

Extremely good results are observed when two anti-CD163 antibodies are paired together (e.g. an antibody of the invention with a second different anti-CD163 antibody), preferably on the same construct. Such bispecific molecules are preferably bi-paratopic constructs in which each different antibody recognises a different epitope (here two different epitopes) on the same antigen (here CD163). Such antibodies are thus sometimes referred to herein as bi-paratopic anti-CD163 antibodies. These bi-paratopic constructs show good inhibition of PRRSV-1 infection, although the effect is reduced in the presence of high concentrations of soluble CD163, and also some inhibition of PRRSV-2 infection.

Even better results are observed when three anti-CD163 antibodies are put together (i.e. an antibody of the invention with a second and a third different anti-CD163 antibody), preferably on the same construct. Such trispecific molecules are preferably tri-paratopic constructs in which each different antibody recognises a different epitope (here three different epitopes) on the same antigen (here CD163). Such antibodies are thus sometimes referred to herein as tri-paratopic anti-CD163 antibodies. Advantageously, the tri-paratopic constructs of the invention can inhibit both PRRSV-1 and PRRSV-2 infection extremely effectively, even in the presence of high concentrations of soluble CD163, such as would likely be found in a pig suffering from infection (or complex (multi-pathogen) disease) as discussed elsewhere herein.

Thus, a preferred embodiment of the invention provides a combination of a membrane specific antibody (or binding protein) of the invention, e.g. the H17B11 antibody as described in Table A, or an antibody comprising three CDRs of SEQ ID NOs: 2, 3 and 4, or sequences substantially homologous thereto, with one or more further anti-CD163 antibodies or binding proteins. Preferably one, two, or more further anti-CD163 antibodies or binding proteins will be used in such combinations. In other words, in total two, three or more different anti-CD163 antibodies (or binding proteins) will be used in combination, wherein one of the antibodies (or binding proteins) is a membrane-specific antibody of the invention.

Where more than one anti-CD163 antibody (or binding protein) is used then preferably each antibody (or binding protein) can bind to a different epitope on CD163 so that all the antibodies (or binding proteins) in the combination can each bind to the CD163 target molecule. Preferably the combinations are provided on the same construct, e.g. joined by appropriate linkers. This format is particularly appropriate for the single domain antibodies (or binding proteins) of the present invention, e.g. the VHH antibodies as described herein.

Preferably such combinations are provided in a single construct. e.g. in a bi-paratopic construct where two (e.g. only two) such antibodies or binding proteins are provided together in a single construct, or a tri-paratopic construct where three (e.g. only three) such antibodies or binding proteins are provided together in a single construct.

A preferred anti-CD163 antibody (or binding protein) for use in combination with a membrane-specific antibody of the invention, in particular a membrane-specific antibody based on the CDR sequences disclosed in Table A, or sequences substantially homologous thereto, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO: 10), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AISWSGRAPYADSVKG (SEQ ID NO:11), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIKWTTLDAYDY (SEQ ID NO:12), or a sequence substantially homologous thereto, wherein said substantially homologous sequence is a sequence containing 1, 2, 3 or 4 amino acid substitutions compared to the given CDR sequence.

In a preferred embodiment, said additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10),
(ii) a variable heavy (VH) CDR2 that comprises the amino acid sequence of AISWSGRAPYADSVKG (SEQ ID NO: 11), and
(iii) a variable heavy (VH) CDR3 that comprises the amino acid sequence of GEGAIKWTTLDAYDY (SEQ ID NO: 12).

Viewed alternatively, a preferred anti-CD163 antibody (or binding protein) for use in combination with a membrane-specific antibody of the invention, in particular a membrane-specific antibody based on the CDR sequences disclosed in Table A, or sequences substantially homologous thereto, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTPSRYV (SEQ ID NO:26), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of ISWSGRA (SEQ ID NO:27), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of AGGEGAIKWTTLDAYDY (SEQ ID NO:28), or a sequence substantially homologous thereto, wherein said substantially homologous sequence is a sequence containing 1, 2, 3 or 4 amino acid substitutions compared to the given CDR sequence.

In a preferred embodiment, said additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTPSRYV (SEQ ID NO:26),
(ii) a variable heavy (VH) CDR2 that comprises the amino acid sequence of ISWSGRA (SEQ ID NO:27), and
(iii) a variable heavy (VH) CDR3 that comprises the amino acid sequence of AGGEGAIKWTTLDAYDY (SEQ ID NO:28).

In certain preferred embodiments of the invention said additional anti-CD163 antibody or binding protein comprises a VH domain that has the amino acid sequence of SEQ ID NO: 9 or a sequence substantially homologous thereto. In some embodiments, such antibodies (or binding proteins) also comprise a VL domain which comprises up to three light chain CDRs, and preferably three light chain CDRs.

In a preferred embodiment said additional anti-CD163 antibody or binding protein comprises a VH domain that has the amino acid sequence of SEQ ID NO: 9, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, said additional anti-CD163 antibody or binding protein comprises a VH domain that has the amino acid sequence of SEQ ID NO: 9. In some embodiments, such antibodies (or binding proteins) also comprise a VL domain which comprises up to three light chain CDRs, and preferably three light chain CDRs.

An exemplary and preferred such antibody is the H03E11 antibody as shown in Table B or D.

Another preferred anti-CD163 antibody (or binding protein) for use in combination with a membrane-specific antibody of the invention, in particular a membrane-specific antibody based on the CDR sequences disclosed in Table A, or sequences substantially homologous thereto, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:19), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:20), or a sequence substantially homologous thereto, wherein said substantially homologous sequence is a sequence containing 1, 2, 3 or 4 amino acid substitutions compared to the given CDR sequence.

In a preferred embodiment, said additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18).
(ii) a variable heavy (VH) CDR2 that comprises the amino acid sequence of CINSITSNTYYADSVKG (SEQ ID NO:19), and
(iii) a variable heavy (VH) CDR3 that comprises the amino acid sequence of DSGLFSGSSCLKYRAMRFGS (SEQ ID NO:20).

Viewed alternatively, another preferred anti-CD163 antibody (or binding protein) for use in combination with a membrane-specific antibody of the invention, in particular a membrane-specific antibody based on the CDR sequences disclosed in Table A, or sequences substantially homologous thereto, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GFTLDDYT (SEQ ID NO:34), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of INSITSNT (SEQ ID NO:35), or a sequence substantially homologous thereto, wherein said substantially homologous sequence is a sequence containing 1, 2, or 3 amino acid substitutions compared to the given CDR sequence, and
(iii) a variable heavy (VH) CDR3 that comprises the amino acid sequence of AADSGLFSGSSCLKYRAMRFGS (SEQ ID NO:36), or a sequence substantially homologous thereto, wherein said substantially homologous sequence is a sequence containing 1, 2, 3 or 4 amino acid substitutions compared to the given CDR sequence.

In a preferred embodiment, said additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GFTLDDYT (SEQ ID NO:34),
(ii) a variable heavy (VH) CDR2 that comprises the amino acid sequence of INSITSNT (SEQ ID NO:35), and
(iii) a variable heavy (VH) CDR3 that comprises the amino acid sequence of AADSGLFSGSSCLKYRAMRFGS (SEQ ID NO:36).

In certain preferred embodiments of the invention said additional anti-CD163 antibody or binding protein comprises a VH domain that has the amino acid sequence of SEQ ID NO:17, or a sequence substantially homologous thereto. In some embodiments, such antibodies (or binding proteins) also comprise a VL domain which comprises up to three light chain CDRs, and preferably three light chain CDRs.

In a preferred embodiment said additional anti-CD163 antibody or binding protein comprises a VH domain that has the amino acid sequence of SEQ ID NO:17, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, said additional anti-CD163 antibody or binding protein comprises a VH domain that has the amino acid sequence of SEQ ID NO:17. In some embodiments, such antibodies (or binding proteins) also comprise a VL domain which comprises up to three light chain CDRs, and preferably three light chain CDRs.

An exemplary and preferred such antibody is the H03D03 antibody as shown in Table C or E.

In combinations where three anti-CD163 antibodies (or binding proteins) are used, the three specific antibodies (or binding proteins) as defined above based on the CDR sequences disclosed in Table A, Table B (or D), and Table C (or E), respectively, or sequences substantially homologous thereto, are a preferred combination, for example in triparatopic constructs of the invention.

In such embodiments, a first preferred anti-CD163 antibody (or binding protein) for use in combination with a membrane-specific antibody of the invention, in particular a membrane-specific antibody based on the CDR sequences disclosed in Table A, or sequences substantially homologous thereto, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO: 10), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AISWSGRAPYADSVKG (SEQ ID NO:11), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIKWTTLDAYDY (SEQ ID NO:12), or a sequence substantially homologous thereto, wherein said substantially homologous sequence is a sequence containing 1, 2, 3 or 4 amino acid substitutions compared to the given CDR sequence.

In a preferred embodiment, said first additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10).
(ii) a variable heavy (VH) CDR2 that comprises the amino acid sequence of AISWSGRAPYADSVKG (SEQ ID NO:11), and
(iii) a variable heavy (VH) CDR3 that comprises the amino acid sequence of GEGAIKWTTLDAYDY (SEQ ID NO:12).

An alternative first additional anti-CD163 antibody (or binding protein) for use in such embodiments, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTPSRYV (SEQ ID NO:26), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of ISWSGRA (SEQ ID NO:27), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of AGGEGAIKWTTLDAYDY (SEQ ID NO:28), or a sequence substantially homologous thereto, wherein said substantially homologous sequence is a sequence containing 1, 2, 3 or 4 amino acid substitutions compared to the given CDR sequence.

In certain preferred embodiments of the invention said first additional anti-CD163 antibody or binding protein comprises a VH domain that has the amino acid sequence of SEQ ID NO: 9 or a sequence substantially homologous thereto. In some embodiments, such antibodies (or binding proteins) also comprise a VL domain which comprises up to three light chain CDRs, and preferably three light chain CDRs.

In a preferred embodiment said first additional anti-CD163 antibody or binding protein comprises a VH domain that has the amino acid sequence of SEQ ID NO: 9, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, said first additional anti-CD163 antibody or binding protein comprises a VH domain that has the amino acid sequence of SEQ ID NO: 9. In some embodiments, such antibodies (or binding proteins) also comprise a VL domain which comprises up to three light chain CDRs, and preferably three light chain CDRs.

An exemplary and preferred such first additional antibody is the H03E11 antibody as shown in Table B or D.

In such embodiments, a second preferred anti-CD163 antibody (or binding protein) for use in combination with the two antibodies (or binding proteins) as outlined above, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:19), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:20), or a sequence substantially homologous thereto, wherein said substantially homologous sequence is a sequence containing 1, 2, 3 or 4 amino acid substitutions compared to the given CDR sequence.

In a preferred embodiment, said second additional anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18),
(ii) a variable heavy (VH) CDR2 that comprises the amino acid sequence of CINSITSNTYYADSVKG (SEQ ID NO:19), and
(iii) a variable heavy (VH) CDR3 that comprises the amino acid sequence of DSGLFSGSSCLKYRAMRFGS (SEQ ID NO:20).

An alternative second additional anti-CD163 antibody (or binding protein) for use in such embodiments, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GFTLDDYT (SEQ ID NO:34), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of INSITSNT (SEQ ID NO:35), or a sequence substantially homologous thereto, wherein said substantially homologous sequence is a sequence containing 1, 2, or 3 amino acid substitutions compared to the given CDR sequence, and
(iii) a variable heavy (VH) CDR3 that comprises the amino acid sequence of AADSGLFSGSSCLKYRAMRFGS (SEQ ID NO:36), or a sequence substantially homologous thereto, wherein said substantially homologous sequence is a sequence containing 1, 2, 3 or 4 amino acid substitutions compared to the given CDR sequence.

In certain preferred embodiments of the invention said second additional anti-CD163 antibody or binding protein comprises a VH domain that has the amino acid sequence of SEQ ID NO: 17, or a sequence substantially homologous thereto. In some embodiments, such antibodies (or binding proteins) also comprise a VL domain which comprises up to three light chain CDRs, and preferably three light chain CDRs.

In a preferred embodiment said second additional anti-CD163 antibody or binding protein comprises a VH domain that has the amino acid sequence of SEQ ID NO:17, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, said second additional anti-CD163 antibody or binding protein comprises a VH domain that has the amino acid sequence of SEQ ID NO:17. In some embodiments, such antibodies (or binding proteins) also comprise a VL domain which comprises up to three light chain CDRs, and preferably three light chain CDRs.

An exemplary and preferred such second additional antibody is the H03D03 antibody as shown in Table C or E.

Binding proteins or constructs or combinations, e.g. binding proteins or constructs or combinations comprising multiple antibodies, based on the antibody sequences set forth in Table A, optionally combined with the antibody sequences set forth in Table B (or D) and/or Table C (or E), are preferred. The invention is exemplified by monoclonal antibodies which are VHH antibodies (single domain antibodies), sequences of which are shown in Tables A, B, C, D and E, herein. The VH CDR domains and VH domains of each of these VHH antibodies are shown in Tables A to E herein. Antibodies (or binding proteins) comprising these sets of VH CDR domains, or VH domains, in particular multi-antibody constructs or binding proteins comprising such domains (or sequences substantially homologous thereto) are preferred embodiments of the invention.

In embodiments where multiple antibodies, e.g. the multiple antibodies described above, are provided in combination on a single construct, the antibodies (or binding proteins) can be provided in any order. Thus, any reference herein to a “first”, “second”, antibody, etc., should not be interpreted to specify where in any construct these antibodies would be positioned. A preferred and exemplified triparatopic construct (Tri-2) comprises (from N-terminus to C-terminus) an antibody based on the sequences of Table B (or D), followed by an antibody based on the sequences of Table C (or E), followed by an antibody based on the sequences of Table A. Another preferred and exemplified triparatopic construct (Tri-10) comprises (from N-terminus to C-terminus) an antibody based on the sequences of Table A, followed by an antibody based on the sequences of Table B (or D), followed by an antibody based on the sequences of Table C (or E). In some embodiments of the invention, the membrane specific antibody of the invention, for example an antibody based on the sequences of Table A, may be positioned at the N terminal or the C-terminal end of the multiple antibodies which are present. Preferred antibodies are VHH antibodies, for example as shown in Tables A to E.

When provided in a single construct, e.g. as a single protein chain, the different antibodies in the construct are conveniently joined by peptide linkers, examples of which are described elsewhere herein.

In an alternative aspect, where two anti-CD163 antibodies (or binding proteins) are used, an anti-CD163 antibody (or binding protein) based on the CDR sequences disclosed in Table B (or D), or sequences substantially homologous thereto, can be combined with an anti-CD163 antibody (or binding protein) based on the CDR sequences disclosed in Table C (or E), or sequences substantially homologous thereto.

Thus, said combinations of antibodies (or binding proteins) or bi-paratopic constructs of this aspect of the invention preferably comprise:
a first antibody (or binding protein) which comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:19), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:20), or a sequence substantially homologous thereto, wherein said substantially homologous sequence is a sequence containing 1, 2, 3 or 4 amino acid substitutions compared to the given CDR sequence; and
a second antibody (or binding protein) which comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AISWSGRAPYADSVKG (SEQ ID NO:11), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIKWTTLDAYDY (SEQ ID NO:12), or a sequence substantially homologous thereto, wherein said substantially homologous sequence is a sequence containing 1, 2, 3 or 4 amino acid substitutions compared to the given CDR sequence.

In an alternative, said first anti-CD163 antibody or binding protein for use in such embodiments, comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GFTLDDYT (SEQ ID NO:34), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of INSITSNT (SEQ ID NO:35), or a sequence substantially homologous thereto, wherein said substantially homologous sequence is a sequence containing 1, 2, or 3 amino acid substitutions compared to the given CDR sequence, and
(iii) a variable heavy (VH) CDR3 that comprises the amino acid sequence of AADSGLFSGSSCLKYRAMRFGS (SEQ ID NO:36), or a sequence substantially homologous thereto, wherein said substantially homologous sequence is a sequence containing 1, 2, 3 or 4 amino acid substitutions compared to the given CDR sequence.

In certain further preferred embodiments said first anti-CD163 antibody or binding protein comprises a VH domain that has the amino acid sequence of SEQ ID NO:17, or a sequence substantially homologous thereto. In some embodiments, such antibodies (or binding proteins) also comprise a VL domain which comprises up to three light chain CDRs, and preferably three light chain CDRs; or
said first anti-CD163 antibody or binding protein comprises a VH domain that has the amino acid sequence of SEQ ID NO:17, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs; or
said first anti-CD163 antibody or binding protein comprises a VH domain that has the amino acid sequence of SEQ ID NO:17. In some embodiments, such antibodies (or binding proteins) also comprise a VL domain which comprises up to three light chain CDRs, and preferably three light chain CDRs.

An exemplary and preferred such antibody is the H03D03 antibody as shown in Table C or E.

In an alternative such embodiment, said second anti-CD163 antibody or binding protein comprises at least one antigen binding domain which binds to CD163, for example porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTPSRYV (SEQ ID NO:26), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of ISWSGRA (SEQ ID NO:27), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of AGGEGAIKWTTLDAYDY (SEQ ID NO:28), or a sequence substantially homologous thereto, wherein said substantially homologous sequence is a sequence containing 1, 2, 3 or 4 amino acid substitutions compared to the given CDR sequence.

In certain preferred embodiments said second additional anti-CD163 antibody or binding protein comprises a VH domain that has the amino acid sequence of SEQ ID NO: 9 or a sequence substantially homologous thereto. In some embodiments, such antibodies (or binding proteins) also comprise a VL domain which comprises up to three light chain CDRs, and preferably three light chain CDRs; or
said second additional anti-CD163 antibody or binding protein comprises a VH domain that has the amino acid sequence of SEQ ID NO: 9, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs; or
said second additional anti-CD163 antibody or binding protein comprises a VH domain that has the amino acid sequence of SEQ ID NO: 9. In some embodiments, such antibodies (or binding proteins) also comprise a VL domain which comprises up to three light chain CDRs, and preferably three light chain CDRs.

An exemplary and preferred such second additional antibody is the H03E11 antibody as shown in Table B or D.

The various antibodies (or binding proteins) or combinations thereof, or constructs comprising said antibodies (or binding proteins) or combinations thereof, described in the above section have the ability to inhibit PRRSV-1 and/or PRRSV-2 infection, and thus can be used in the treatment or prevention of PRRSV-1 and/or PRRSV-2 infection.

As a preferred use of the antibody (or binding protein) containing constructs or combinations of the invention is in the treatment or prevention of pathogenic infections which involve CD163, most notably PRRSV infection, typically, antibody (or binding protein) containing constructs and combinations of the invention inhibit (or block or reduce) pathogen (e.g. PRRSV) infection, for example inhibit (or block or reduce) the ability of the pathogen, e.g. PRRSV, to cause infection (e.g. to infect appropriate host cells). Preferably, the inhibition or reduction is a measurable inhibition or reduction, more preferably a significant inhibition or reduction, e.g. a statistically significant inhibition or reduction such as with a probability value of ≤0.05 or <0.05. In certain embodiments, antibody (or binding protein) containing constructs or combinations of the invention can inhibit (or block or reduce) the ability of the pathogen, e.g. PRRSV, to infect host cells by at least 20%, at least 25%, at least 30%, at least 40%, at least 50%, at least 60%, at least 70%, at least 75%, at least 80%, at least 85%, at least 90%, at least 92%, at least 95%, or at least 98%, e.g. complete or 100% inhibition. Typically, such % inhibition (and other percentage inhibition levels as described herein) is in comparison with (or relative to) an appropriate control assay or control level, for example a control assay or control level in the absence of a binding protein or antibody (anti-CD163 antibody) (for example a negative control or background level or assay). Thus, a 0% inhibition (control) level (or conversely a 100% or maximum infection level) is typically the level in the absence of a binding protein or antibody (anti-CD163 antibody).

Such ability to inhibit infection can be determined or tested in any appropriate assay, examples of which would be readily derived by a person skilled in the art. Appropriate assays might for example be in vitro or ex vivo assays and for example involve the use of CD163 expressing host cells such as PAMs or recombinant CD163 expressing host cells as discussed elsewhere herein. Such cells can be brought into contact with PRRSV or other appropriate pathogens at a level which will cause infection of the cells. Appropriate assays may typically be carried out in the presence of serum, e.g. porcine serum (from either healthy or infected pigs, see e.g. below, or serum which otherwise contains soluble CD163) or fetal bovine serum (FBS), or in the presence of soluble CD163. The appropriate percentage of serum to use is readily determined by a skilled person, for example levels of 10% FBS and 80% porcine serum were used in the assays described in the Examples section.

FBS does not generally contain any soluble CD163. However, porcine serum does typically contain at least some soluble CD163. Thus, in order to test the ability of the antibody (or binding protein) containing constructs or combinations of the invention to inhibit PRRSV infection in the presence of soluble CD163, the use of porcine serum, typically 80% porcine serum, was used. The level of soluble CD163 is typically significantly elevated in pigs suffering from an infection, e.g. pigs undergoing an inflammatory response. Thus, in order to test the ability of the antibody (or binding protein) containing constructs or combinations of the invention to inhibit PRRSV infection in the presence of high (but physiological) levels of soluble CD163, the use of porcine serum from infected pigs (e.g. Lawsonia intracellularis infected pigs), typically 80% porcine serum, was used. Equally serum from pigs infected with other bacteria or viruses such that levels of soluble CD163 are elevated could be used. Methods of determining the level of soluble CD163 in a relevant serum sample could routinely be carried out to establish the level of soluble CD163 present. In the porcine serum assays described herein, the level of soluble CD163 in the assays using serum from healthy pigs was determined to be 0.40 mg/l and in infected pigs it was 4.50 mg/l. Thus, such levels of soluble CD163 are exemplary and preferred and in some embodiments of the invention the levels of inhibition described herein are levels observed in the presence of those concentrations of soluble CD163.

Ability of the antibody (or binding protein) containing constructs or combinations of the invention to inhibit or reduce such infection can then readily be analysed, for example in comparison with (or relative to) a 100% infection level set by the control assay. An appropriate and exemplary infection assay is described in the Examples section.

Any appropriate concentrations of the antibody (or binding protein) containing constructs or combinations of the invention may be used to inhibit or reduce infection. Exemplary constructs or combinations of the invention have the ability to cause inhibition, e.g. the levels of inhibition as outlined herein, with antibody, in particular VHH, when used at concentrations of at least 1, 2, 4, 5, 8, 10, 20, 30, 40, 50, 60, 70, 80, 90, 100, 120, 140, 160, 180, 200, 300 or 400 μg/ml, e.g. at concentrations up to 200, 300 or 400 μg/ml, e.g. between 50 or 100 and 200, 300 or 400 μg/ml. Preferred constructs or combinations of the invention have the ability to cause inhibition, e.g. the levels of inhibition as outlined herein, with antibody, in particular VHH, when used at concentrations of or at least 50, 60, 70, 80, 90, 100, 120, 140, 160, 170, 180, 190, 200, 225, 250, 275, 300, 325, 350, 375, 400, 450, 500, 600, 700, 800 or 900 ng/ml, e.g. at concentrations up to 100, 200, 300, 400, 450, 500, 600, 700, 800 or 900 ng/ml, e.g. between 50 or 100 and 200, 300, 400 or 500 ng/ml. If combinations of antibodies (e.g. VHH antibodies) are used then these levels in some embodiments can refer to the total amount of antibody (e.g. VHH) present, i.e. the sum of the individual concentrations of antibodies present.

Exemplary constructs or combinations of the invention have the ability to cause inhibition, e.g. the levels of inhibition as outlined herein, with antibody, in particular VHH, when used at concentrations of or at least 5, 10, 15, 20, 25, or 30 μM. Preferred constructs or combinations of the invention have the ability to cause inhibition, e.g. the levels of inhibition as outlined herein, with antibody, in particular VHH, when used at concentrations of or at least 50, 60, 70, 80, 90, 100, 120, 140, 160, 170, 180, 190, 200, 250, 275, 300, 325, 350, 375, 400, 500, 600, 700, 800 or 900 nM, e.g. at concentrations up to 100, 200, 300, 400, 500, 600, 700, 800 or 900 nM, e.g. between 50 or 100 and 200, 300, 400 or 500 nM. More preferred constructs of the invention have the ability to cause inhibition, e.g. the levels of inhibition as outlined herein, with antibody, in particular VHH, when used at concentrations of or at least 1, 2, 3, 4, 5, 6, 7, 8, 9, 10, 15, 20, 25, 30, 35, 40, 45 or 50 nM, e.g. at concentrations up to 10, 20, 30, 40 or 50 nM, e.g. between 5 or 10 and 30, 40, or 50 nM. If combinations of antibodies (e.g. VHH antibodies) are used then these levels in some embodiments can refer to the total amount of antibody (e.g. VHH) present, i.e. the sum of the individual concentrations of antibodies present.

In some embodiments, the antibody (or binding protein) containing constructs or combinations of the invention can inhibit (or block or reduce) the ability of PRRSV-1 or PRRSV-2 to cause infection (e.g. to infect CD163 expressing host cells). In some embodiments, the binding protein or antibody of the invention can inhibit (or block or reduce) the ability of both PRRSV-1 and PRRSV-2 to cause infection (e.g. to infect CD163 expressing host cells). It can be noted that the antibody (or binding protein) containing constructs or combinations of the invention targets host cell CD163 as opposed to the PRRSV (or other pathogenic entity) per se. This provides an important advantage of being able to inhibit infection by any virus, e.g. PRRSV, which uses the same binding region on CD163 for infection or pathogenesis. In this way, the antibodies, etc., of the invention can provide a means for blocking many strains or isolates of PRRSV, including high pathogenic strains or isolates, providing they use CD163 in order to infect cells. It is believed that CD163 utilisation is common for infection by multiple PRRSV strains. Thus, the constructs and combinations of the present invention have wide utility. This is in contrast to for example some of the known approaches for PRRSV, e.g. vaccination, which can be strain specific, and their effectiveness (or whether they are effective at all) can vary depending on the strain. Thus, the constructs or combinations of the invention provide important advantages and flexibility over such prior methods.

Preferred constructs or combinations of the invention have the ability to almost completely inhibit, or completely inhibit, PRRSV-1 infection, for example at least 90%, 95% or 98% inhibition can be observed or 100% inhibition can be observed. Alternatively, at least 50%, 60%, 70%, 75% or 80% inhibition can be observed. In some embodiments antibodies which have the ability to show at least 80% inhibition of PRRSV-1 infection, more preferably at least 85%, 90%, 95% or 98% inhibition are preferred.

Preferred constructs or combinations of the invention have the ability to almost completely inhibit, or completely inhibit, PRRSV-2 infection, for example at least 90%, 95% or 98% inhibition can be observed or 100% inhibition can be observed. Alternatively, at least 50%, 60%, 70%, 75% or 80% inhibition can be observed. In some embodiments antibodies which have the ability to show at least 80% inhibition of PRRSV-2 infection, more preferably at least 85%, 90%, 95% or 98% inhibition are preferred. PRRSV-2

Other preferred constructs or combinations of the invention have the ability to inhibit PRRSV-1 and/or PRRSV-2 infection by at least 40%, at least 45%, at least 50%, at least 55%, at least 60%, or at least 65%, more preferably by at least 70%, 80%, 85%, 90%, 95% or 98% in the presence of soluble CD163, e.g. in the presence of relatively low levels of soluble CD163, for example levels of up to or at or around 0.40 mg/L.

Other preferred constructs or combinations of the invention have the ability to inhibit PRRSV-1 and/or PRRSV-2 infection by at least 40%, at least 45%, at least 50%, at least 55%, at least 60%, or at least 65%, more preferably by at least 70%, 80%, 85%, 90%, 95% or 98% in the presence of soluble CD163, e.g. in the presence of relatively high levels of soluble CD163, for example levels of up to or at or around 4.50 mg/L.

In some embodiments, the constructs or combinations of the invention can inhibit (or block or reduce) the ability of the PRRSV-2 to infect host cells. In some embodiments, the binding protein or antibody of the invention has the ability to specifically inhibit (or block or reduce) the ability of the PRRSV-2 to cause infection (e.g. to infect CD163 expressing host cells or specifically inhibit PRRSV-2 infection). Exemplary antibodies may therefore be capable of at least 25%, 30%, 35%, 40%, 45% or 50% inhibition of PRRSV-2 infection (e.g. inhibit the ability of PRRSV-2 to infect host cells by at least 25%, 30%, 35%, 40%, 45% or 50%).

Exemplary constructs or combinations comprise the three VHH antibodies as shown in Tables A, B (or D), and C (or E).

In certain embodiments, constructs or combinations of the present invention have an IC₅₀ (e.g. for the inhibition of PRRSV1 and/or PRRSV2 infection of host cells, e.g. PAMs) of 10.0 nM or less, 9.5 nM or less, 9.0 nM or less, 8.5 nM or less, 8.0 nM or less, 7.5 nM or less, 7.0 nM or less, 6.5 nM or less, 6.0 nM or less, 5.5 nM or less, 5 nM or less, 4.5 nM or less, 4.0 nM or less, 3.5 nM or less, or 3.0 nM or less. In some embodiments, the IC₅₀ is 2.0, 2.5, 3.0 or 3.5 to 5.0, 5.5, 6.0, 6.5, 7.0, 7.5, 8.0, 8.5, 9.0, 9.5 or 10.0 nM. Particular exemplary IC₅₀ values are also shown in the Examples.

In certain embodiments, constructs or combinations of the present invention have an IC₉₀ (e.g. for the inhibition of PRRSV1 and/or PRRSV2 infection of host cells, e.g. PAMs) of 30.0 nM or less, 25.0 nM or less, 20.0 nM or less, 15.0 nM or less, 12 nM or less, 10.0 nM or less, 9.5 nM or less, 9.0 nM or less. 8.5 nM or less, 8.0 nM or less, 7.5 nM or less, 7.0 nM or less, 6.5 nM or less, 6.0 nM or less, 5.5 nM or less, 5 nM or less, 4.5 nM or less. 4.0 nM or less, 3.5 nM or less, or 3.0 nM or less. In some embodiments, the IC₉₀ is 2.0, 2.5, 3.0 or 3.5 to 5.0, 5.5, 6.0, 6.5, 7.0, 7.5, 8.0, 8.5, 9.0, 9.5, 10.0, 15.0, 20.0, 25.0 or 30.0 nM. Particular exemplary IC₉₀ values are also shown in the Examples.

The preferred IC₅₀ or IC₉₀ values as described above are preferably as determined in an appropriate virus infectivity assay, e.g. as described above or in the Examples section.

In alternative embodiments of the invention, the constructs or combinations of the invention can be used to reduce the risk of or prevent PRRSV infection.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better (preferably statistically significantly better), when compared to the abilities observed for prior art antibodies.

In any statistical analysis referred to herein, preferably the statistically significant difference over a relevant control or other comparative entity or measurement has a probability value of ≤0.1 or <0.1, preferably ≤0.05 or <0.05. Appropriate methods of determining statistical significance are well known and documented in the art and any of these may be used.

In some embodiments, constructs or combinations of the present invention have one or more, preferably two or more, or three or more, most preferably all, of the functional properties, in particular the preferred functional properties, described herein.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, by way of example, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

Nucleic acid molecules comprising nucleotide sequences that encode the binding proteins or antibodies of (or used in) the present invention, or that encode constructs or combinations as defined herein, or parts or fragments thereof, or nucleic acid molecules substantially homologous thereto, form yet further aspects of the invention.

Preferred nucleic acid molecules are those encoding a VHH antibody or a VH region or domain of the present invention (e.g., those encoding SEQ ID NO:1, 9, or 17). Other preferred nucleic acid molecules are those encoding the sets of three CDR sequences as defined in any one of Tables A, B, C, D or E, or sequences substantially homologous thereto. Preferred such nucleic acid molecules also encode appropriate framework regions, e.g. FR1, FR2, FR3 and FR4 regions, preferably the sets of FR sequences as defined in any one of Tables A, B, C, D or E, or sequences substantially homologous thereto.

Nucleic acid molecules of the invention may for example be DNA or RNA molecules.

The term “substantially homologous” as used herein in connection with an amino acid or nucleic acid sequence includes sequences having at least 55%, 60%, 65%, 70% or 75%, preferably at least 80%, and even more preferably at least 85%, 90%, 95%, 96%, 97%, 98% or 99%, sequence identity to the amino acid or nucleic acid sequence disclosed. In certain embodiments, the antibodies (or binding proteins) of the invention comprise one or at least one heavy chain variable region (or VH domain) that includes an amino acid sequence region of at least about 55%, 60%, 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO: 1, 9, and 17.

Substantially homologous sequences of the invention thus include single or multiple base or amino acid alterations (additions, substitutions, insertions or deletions) to the sequences of the invention. At the amino acid level preferred substantially homologous sequences contain up to 5, e.g. only 1, 2, 3, 4 or 5, preferably 1, 2, 3 or 4, preferably 1, 2 or 3, more preferably 1 or 2, altered amino acids, in one or more of the framework regions and/or one or more of the CDRs making up the sequences of the invention. Said alterations can be with conservative or non-conservative amino acids. Preferably said alterations are substitutions, preferably conservative amino acid substitutions.

In certain embodiments, if a given starting sequence is relatively short (e.g. five amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which in some embodiments may be five amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the individual CDRs, for example a sequence identity of at least 50%, 55%, 60%, 70%, 75%, 80%, 85%, 90%, 95%, 96%, 97%, 98% or 99%. Thus, by way of example, in CDR sequences herein which are 20 or 22 amino acids in length, up to 8, e.g. only 1, 2, 3, 4, 5, 6, 7 or 8, or 1, 2, 3, 4, 5, 6 or 7, or 1, 2, 3, 4, 5 or 6, or 1, 2, 3, 4 or 5, preferably 1, 2, 3 or 4, preferably 1, 2 or 3, more preferably 1 or 2, altered amino acids, may be present.

Other preferred examples of substantially homologous sequences are sequences having at least 50%, at least 55%, at least 60%, at least 65%, at least 70%, at least 75%, at least 80%, at least 85%, at least 90%, or at least 95%, amino acid sequence identity to the amino acid sequence of one or more of the CDR regions or one or more of the FR regions disclosed in Tables A or B or C or D or E. Thus, in some embodiments, a substantially homologous CDR sequence may be a sequence having at least 50%, at least 55%, at least 60%, at least 65%, at least 70%, at least 75%, at least 80%, at least 85%, at least 90% or at least 95% sequence identity to a given CDR sequence described herein.

In some embodiments, in antibodies having a substantially homologous sequence as compared to a given sequence, or having a certain degree of sequence identity as compared to a given sequence, the altered amino acid residues(s) are not in a CDR region. For example, in some embodiments, in antibodies having a VH domain that has a certain degree of sequence identity to a given VH domain sequence of a particular antibody of (or used in) the invention (e.g. as disclosed in Tables A, B or C, D or E), the altered (or variant) residue(s) are not in a CDR region. Thus, in some embodiments, in antibodies having a substantially homologous sequence as compared to a given sequence, or having a certain degree of sequence identity as compared to a given sequence, the altered amino acid residues(s) are in one or more framework regions.

As is evident from elsewhere herein, in other embodiments, in antibodies having a substantially homologous sequence as compared to a given sequence, or having a certain degree of sequence identity as compared to a given sequence, the altered amino acid residues(s) may be in a CDR region.

In some embodiments, in an antibody (or binding protein) having a substantially homologous sequence as compared to a given sequence, or having a certain degree of sequence identity as compared to a given sequence, the three VH CDR amino acid sequences (i.e. all three VH CDR sequences taken together) are considered together to be the whole (or entire) CDR complement of the antibody, and the amino acid sequence of said whole CDR complement of said antibody is at least 60%, 65%, or 70%, preferably at least 75% or 80%, or at least 85% or 90%, or at least 95% identical to the corresponding whole (or entire) CDR complement of a given starting (or reference) antibody. The starting (or reference) antibody may have the CDR sequences of the antibodies disclosed in Tables A, B or C, D or E.

Altered residues might be conserved or non-conserved amino acid substitutions, or a mixture thereof. In such embodiments, preferred alterations are conservative amino acid substitutions.

In all embodiments, binding proteins, e.g. antibodies, containing substantially homologous sequences retain the ability to bind to CD163, e.g. porcine CD163. Preferably, binding proteins, e.g. antibodies, containing substantially homologous sequences retain one or more (preferably all) of the other properties described herein in relation to the H17B11 (Table A), H03E11 (Table B) or H03D03 (Table C) antibodies, as appropriate.

The CDRs of the antibodies (or binding proteins) of the invention are preferably separated by appropriate framework regions such as those found in naturally occurring antibodies and/or effective engineered antibodies. Thus, the V_{H} (e.g. VHH), V_{L} and individual CDR sequences of the invention are preferably provided within or incorporated into an appropriate framework or scaffold to enable antigen (here CD163) binding. Such framework sequences or regions may correspond to naturally occurring framework regions, FR1, FR2, FR3 and/or FR4, as appropriate to form an appropriate scaffold, or May 20 correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Preferred sequences for framework regions are one or more of the framework regions making up the VHH antibodies of (or used in) the invention, preferably one or more of the framework regions of the H17B11, H03E11, or H03D03 VHH antibodies, as disclosed in Tables A, B (or D) and C (or E), respectively, or framework regions substantially homologous thereto, and in particular framework regions that allow the maintenance of antigen specificity, for example framework regions that result in substantially the same or the same 3D structure of the antibody.

In certain preferred embodiments, all four of the variable heavy chain (SEQ ID NOs: 5, 6, 7 and 8) framework regions (FR), as appropriate, or FR regions substantially homologous thereto, are found in the antibodies (or binding proteins) of the invention, in particular for antibodies (or binding proteins) based on the CDRs of SEQ ID NOs: 2, 3 and 4.

In other preferred embodiments, all four of the variable heavy chain (SEQ ID NOs:13, 14, 15 and 16) framework regions (FR), as appropriate, or FR regions substantially homologous thereto, are found in the antibodies (or binding proteins) of the invention, in particular for antibodies (or binding proteins) based on the CDRs of SEQ ID NOs: 10, 11 and 12.

In other preferred embodiments, all four of the variable heavy chain (SEQ ID NOs: 21, 22, 23 and 24) framework regions (FR), as appropriate, or FR regions substantially homologous thereto, are found in the antibodies (or binding proteins) of the invention, in particular for antibodies (or binding proteins) based on the CDRs of SEQ ID NOs: 18, 19 and 20.

In other preferred embodiments, all four of the variable heavy chain (SEQ ID NOs: 29, 30, 31 and 32) framework regions (FR), as appropriate, or FR regions substantially homologous thereto, are found in the antibodies (or binding proteins) of the invention, in particular for antibodies (or binding proteins) based on the CDRs of SEQ ID NOs: 26, 27 and 28.

In other preferred embodiments, all four of the variable heavy chain (SEQ ID NOs: 37, 38, 39 and 40) framework regions (FR), as appropriate, or FR regions substantially homologous thereto, are found in the antibodies (or binding proteins) of the invention, in particular for antibodies (or binding proteins) based on the CDRs of SEQ ID NOs: 34, 35 and 36.

CDR sequences of certain antibodies of the invention are set forth herein in Tables A, B, C, D and E. In some embodiments, CDR sequences of antibodies of the invention may be CDR sequences in the VH (VHH) domains of antibodies of the invention as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, MD, 647-669, 1991) or Chothia (e.g. Chothia C, et al. (1989) Nature, 342:877-883, or Al-Lazikani et al., (1997) JMB 273, 927-948), or as identified using the IMGT numbering scheme (e.g. Lefranc, M.-P., The Immunologist, 7, 132-136 (1999); www.imgt.org)).

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antibody to form a new antibody, wherein said parent antibody is one of the antibodies of the invention as defined elsewhere herein, and testing the resulting new antibody to identify antibodies that bind to CD163 in accordance with the invention, for example membrane-specific antibodies as described elsewhere herein, can be carried out using techniques which are routine in the art. Such methods can be used to form multiple new antibodies that can all be tested for their ability to bind CD163, and for example their ability to show membrane-specific binding as described elsewhere herein. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way. Testing the ability of one or more of the modified antibodies to bind to CD163, and for example their ability to show membrane-specific binding as described elsewhere herein, can be carried out by any appropriate method, which are well known and described in the art. Suitable methods are also described elsewhere herein and in the Examples section.

New antibodies produced, obtained or obtainable by these methods form a yet further aspect of the invention.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antibody should retain the ability to bind to CD163 as described above. Preferably, any substantially homologous antibody should retain one or more (or all) of the functional capabilities of the starting antibody, for example, if appropriate, the ability to show membrane-specific binding as described elsewhere herein.

Preferably, any substantially homologous antibody should retain the ability to specifically bind to the same epitope of CD163 as recognized by the starting antibody in question, for example, the same epitope recognized by the CDR domains of one or more of the antibodies of the invention or the VH (VHH) domains of the invention as described herein, e.g. bind to the same epitope as one or more of the various antibodies of the invention (e.g. one or more of the VHH antibodies as shown in Tables A, B, C, D or E). Thus, preferably, any substantially homologous antibody should retain the ability to compete, in a suitable assay, with one or more of the various antibodies of the invention (e.g. VHH antibodies as shown in Tables A, B, C, D or E, as appropriate) for binding to CD163.

Binding to the same epitope/antigen can be readily tested by methods well known and described in the art, e.g. using binding assays, e.g. a competition assay or by analysis of the crystal structure of the antigen-antibody complex. Retention of other functional properties can also readily be tested by methods well known and described in the art or herein.

Thus, a person skilled in the art will appreciate that binding assays can be used to test whether any antibodies, for example “substantially homologous” antibodies, have the same binding specificities, e.g. bind to the same epitope, or with the same or equivalent affinity, as the antibodies and antibody fragments of the invention, for example, binding assays such as competition assays or ELISA assays as described elsewhere herein. For example, assays which can measure the binding of antibodies to cells expressing CD163, such as FACS assays, are preferred to determine if an antibody (or binding protein) binds to the membrane-bound form of CD163, e.g. porcine CD163. SPR, e.g. BIAcore assays could also readily be used to establish whether antibodies, for example “substantially homologous” antibodies, can bind to CD163, e.g. can bind (or not) to soluble forms of CD163. Indeed SPR (BIAcore) assays are preferred to determine if an antibody (or binding protein) does not bind or does not bind significantly to the soluble form of CD163, e.g. porcine CD163. The skilled person will be aware of other suitable methods and variations.

As outlined below, a competition binding assay can be used to test whether antibodies, for example “substantially homologous” antibodies retain the ability to specifically bind to substantially the same epitope of CD163 as recognized by one or more of the antibodies of the invention as shown in the various sequence Tables herein, or have the ability to compete with one or more of the various antibodies of the invention as shown in the various sequence Tables herein. The method described below is only one example of a suitable competition assay. The skilled person will be aware of other suitable methods and variations.

An exemplary competition assay involves assessing the binding of various effective concentrations of an antibody of the invention to CD163 (for example membrane-bound forms of CD163) in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to CD163, for example membrane-bound forms of CD163) is evidence of binding to substantially the same epitope. Preferably, the test antibody significantly reduces the amount of antibody of the invention that binds to CD163 (for example membrane-bound forms of CD163). Preferably, the test antibody reduces the amount of antibody of the invention that binds to CD163 (for example membrane-bound forms of CD163) by at least about 95%. ELISA and flow cytometry assays may be used for assessing inhibition of binding in such a competition assay but other suitable techniques would be well known to a person skilled in the art. Flow cytometry assays are particularly preferred when membrane-specific antibodies of the invention are concerned.

Such antibodies (monoclonal antibodies) which have the ability to specifically bind to substantially the same (or the same) epitope of CD163 or an overlapping epitope of CD163 as recognized by the antibodies of the invention, for example the membrane-specific antibodies of the invention (e.g. the VHH antibody H17B11 as shown in Table A) or which have the ability to compete with the antibodies of the invention, for example the membrane-specific antibodies of the invention (e.g. the VHH antibody H17B11 as shown in Table A) are further embodiments of the present invention.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to CD163, e.g. membrane-bound forms of CD163. Preferably, the competing antibody can bind to the same epitope as the reference antibody. Alternatively viewed, the competing antibody preferably has the same epitope specificity as the reference antibody.

“Reference antibodies” as used herein are antibodies which can bind to CD163 in accordance with the invention which preferably have a VH domain as defined herein, more preferably have a VH domain or are a VHH antibody comprising SEQ ID NO: 1, 9, or 17 (or the relevant three CDR sequences of said sequences) as outlined in Tables A, B, C, D or E. A preferred reference antibody has a VH domain or is a VHH antibody comprising SEQ ID NO: 1 (or the relevant three CDR sequences of said sequence) as outlined in Table A. In other words a preferred reference antibody is the membrane-specific antibody as defined in Table A.

The identification of one or more competing antibodies or antibodies that bind to the same epitope is a straightforward technical matter now that reference antibodies such as those outlined in the sequence Tables herein have been provided. In particular, the identification of one or more competing antibodies that bind to the same epitope as the VHH antibody of Table A, and show membrane-specific binding activity, is preferred. As the identification of competing antibodies or antibodies that bind to the same epitope can be determined in comparison to a reference antibody, it will be understood that actually determining the epitope to which either or both antibodies bind is not in any way required in order to identify a competing antibody or an antibody that binds to the same epitope. However, epitope mapping can be performed using standard techniques, if desired.

Thus, a yet further aspect provides an antibody (or binding protein) comprising an antigen binding domain which binds or specifically binds to CD163, e.g. porcine CD163, wherein said antibody (antigen binding domain) binds to the same epitope as the VHH antibody of Table A (or an antibody with the CDRs as defined in Table A, or CDRs substantially homologous thereto), and shows membrane-specific binding activity as described elsewhere herein.

To the inventors' knowledge, antibodies (e.g. monoclonal antibodies) which can bind (or specifically bind) to porcine CD163, wherein said antibodies bind to the membrane-bound form of porcine CD163 on cells, but do not bind significantly to the soluble form of porcine CD163 as described herein, have not been described in the art, but are described herein. Preferably said antibodies bind within the SRCR5 domain of CD163.

Thus, the individual membrane-specific monoclonal antibodies as described herein, e.g. in Table A, are both unusual and advantageous. In addition, the epitope bound by such antibodies, and antibodies which bind to this same epitope, are also of interest. Thus it is believed that the antibodies of the present invention can bind to a novel epitope, e.g. a conformational epitope, in the SRCR5 region of porcine CD163 on cells that can confer membrane-specific binding.

Substantially homologous sequences of proteins of the invention include, without limitation, conservative amino acid substitutions, or for example alterations that do not affect the VH, VL or CDR domains of the antibodies, e.g. antibodies where tag sequences, toxins or other components are added that do not contribute to the binding of antigen, or alterations to convert one type or format of binding protein, antibody molecule or fragment to another type or format of binding protein, antibody molecule or fragment (e.g. conversion from VHH to Fab or scFv or whole antibody or vice versa), or the conversion of an antibody molecule to a particular class or subclass of antibody molecule (e.g. the conversion of an antibody molecule to IgG or a subclass thereof, e.g. IgG₂).

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Homology may be assessed by any convenient method. However, for determining the degree of homology between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 60%, 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

The terms “antibody” and “immunoglobulin”, as used herein, refer broadly to any immunological binding agent that comprises an antigen binding domain, including polyclonal and monoclonal antibodies. Monoclonal antibodies are however preferred. In other words, in some embodiments antibodies of the invention are not polyclonal antibodies. Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like, for example camelid antibodies are IgG antibodies which often have IgG2 or IgG3 constant domains. The heavy-chain constant domains that correspond to the difference classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

The term “heavy chain complementarity determining region” (“heavy chain CDR”) as used herein refers to regions of hypervariability within the heavy chain variable region (V_{H} domain) of an antibody molecule or within a VHH antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (V_{H} domain) as used herein refers to the variable region of a heavy chain of an antibody molecule.

The term “light chain complementarity determining region” (“light chain CDR”) as used herein refers to regions of hypervariability within the light chain variable region (V_{L} domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region (e.g. an antigen binding region comprising CDRs and optionally FRs derived from an antibody molecule, or corresponding thereto), and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BITE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In all embodiments of the invention, single domain antibodies (also referred to as VHH antibodies, sdAbs, DABs, dAbs, nanobodies, camelid antibodies, vNAR (shark) antibodies, VH antibodies or VL antibodies) are preferred, in particular VHH antibodies, nanobodies, camelid antibodies, and vNAR (shark) antibodies. Such antibodies comprise a single monomeric variable antibody domain, usually a VH domain, which can bind to antigen (although single VL domains which have the ability to bind antigen have been described and can be used). Thus, in some such preferred embodiments the antibodies (or antigen binding domains) of the invention comprise (or consist of) one (or a single or only a single or one or only one) heavy chain variable region (VH or VHH), although in some embodiments a number of these individual heavy chain variable regions with the same or different sequences can be present together in the same construct or molecule.

Such antibodies can be obtained or prepared using standard techniques which are well known and described in the art. For example, such antibodies can be obtained by immunizing appropriate animals, e.g. camelids such llamas, or sharks, with the desired antigen and then cloning the VH domains of the antibodies generated into appropriate expression vectors and selecting for binders. Libraries of VH domains (e.g. phage display libraries of human VH domains) are also available or can be generated and can then be screened.

Due to their relatively small size, single domain antibodies can have a relatively short half life, e.g. a relatively short plasma half life. Thus, such antibodies are sometimes modified in order to extend or prolong their half life and such modified antibodies (binding proteins) form part of the present invention. Techniques to do this are well known and described in the art and any of these may be used. Examples include attaching or conjugating or fusing the antibodies (binding proteins) to albumin/serum albumin (or another protein or entity which itself has a long (or longer) half life, e.g. a longer half life than the antibody to which the protein or entity is fused, or an alternative entity or protein that can act to extend the half life of proteins (e.g. antibodies) to which it is attached), or attaching or conjugating or fusing the antibodies (binding proteins) to another protein or entity (e.g. an antibody, e.g. a VHH antibody) which can itself interact with a protein or entity which has a long (or longer) half life (e.g. attaching etc., to an antibody, e.g. a VHH antibody, that binds to IgG, e.g. porcine IgG), or attaching or conjugating the antibodies (binding proteins) to PEG (or other polymers, e.g. hydrophilic polymers), or attaching or conjugating or fusing the antibodies (binding proteins) to an antibody, or other protein or entity, which binds to FcRn. In this regard, fusion to an IgG Fc region is an established strategy to extend the half life of therapeutic proteins. Thus, preferred antibodies (binding proteins) comprise an Fc region or domain, e.g. are fused to an Fc region or domain (in other words are Fc fusions). Such Fc regions or domains are known in the art and generally comprise CH2 and CH3 domains of antibody heavy chains, which associate to form a homodimer. These regions can be derived from any appropriate source or species, e.g. a source or species different from the host species used to generate the antibodies, e.g. by immunization, or a source or species different from where the antibodies are derived, but preferably correspond to or are derived from porcine Fc regions or domains.

Thus, in some embodiments of the invention, the antibodies (or binding proteins) comprise, or are joined or otherwise fused to, an entity that can extend half life, preferably albumin or an IgG Fc region.

In some embodiments of the invention, the antibodies (or binding proteins) comprise, or are joined or otherwise fused to, an entity that can extend half life, for example a further antibody, e.g. a VHH antibody, that can extend half life.

In certain embodiments, the antibody or antibody fragment of the present invention comprises all or a portion of a heavy chain constant region, such as an IgG1, IgG2, IgG3, IgG4, IgA1, IgA2, IgE, IgM or IgD constant region. Preferably, the heavy chain constant region is an IgG heavy chain constant region, e.g. an IgG2 heavy chain constant region, or a portion thereof. Furthermore, the antibody or antibody fragment can comprise all or a portion of a kappa light chain constant region or a lambda light chain constant region, or a portion thereof. All or part of such constant regions may be produced naturally or may be wholly or partially synthetic. Appropriate sequences for such constant regions are well known and documented in the art. When a full complement of constant regions from the heavy and light chains are included in the antibodies of the invention, such antibodies are typically referred to herein as “full length” antibodies or “whole” antibodies. In some embodiments, IgG₂ antibodies are preferred.

In other embodiments it is preferred that no constant regions, e.g. no heavy chain or light chain constant regions, are present, e.g. a variable domain or heavy chain variable domain (VH) is the only part of an antibody that is present.

The antibodies or antibody fragments can be produced naturally or can be wholly or partially synthetically produced.

Many antibodies or antibody fragments comprise an antibody light chain variable region (V_{L}) that comprises three CDR domains and an antibody heavy chain variable region (V_{H}) that comprises three CDR domains. Said VL and VH generally form the antigen binding site.

However, it is well documented in the art that the presence of three CDRs from the light chain variable domain and three CDRs from the heavy chain variable domain of an antibody is not always necessary for antigen binding. Thus, constructs smaller than the above classical antibody fragment are known to be effective.

For example, camelid antibodies have an extensive antigen binding repertoire but are devoid of light chains. Also, results with single domain antibodies comprising VH domains alone or VL domains alone show that these domains can bind to antigen with acceptably high affinities and have other advantages such as their small size and ease of production. Thus, three CDRs can effectively bind antigen and such single domain antibodies as described and exemplified herein (e.g. a VHH antibody), are preferred.

The antibody, binding protein and nucleic acid molecules of the invention are generally “isolated” or “purified” molecules insofar as they are distinguished from any such components that may be present in situ within a human or animal body (e.g. a camelid) or a tissue sample derived from a human or animal body (e.g. a camelid). The sequences may, however, correspond to or be substantially homologous to sequences as found in a human or animal body (e.g. a camelid). Thus, the term “isolated” or “purified” as used herein in reference to nucleic acid molecules or sequences and proteins or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

It can be noted that the antibodies etc., of the invention do not occur in nature and are, in that respect, man-made constructs in that they do not correspond to molecules that occur naturally. For example, preferred antibodies are single domain antibodies which can be engineered or recombinantly produced, and even in species that produce such antibodies naturally, e.g. camelids, such species will not produce antibodies to CD163, in particular porcine CD163, unless they are experimentally induced to do so, e.g. by immunization. In other words the antibodies, etc., of the invention are non-native.

The term “fragment” as used herein refers to fragments of biological relevance, e.g. fragments that contribute to antigen binding, e.g. form part of the antigen binding site, and/or contribute to the functional properties of the CD163 antibody. Certain preferred fragments comprise or consist of a heavy chain variable region (V_{H} domain or the three VH CDRs) of the antibodies of the invention.

A person skilled in the art will appreciate that the proteins and polypeptides of the invention, such as the heavy and light chain CDRs, the heavy and light chain variable regions, antibodies and antibody fragments, may be prepared in any of several ways well known and described in the art, but are most preferably prepared using recombinant methods.

Nucleic acid fragments encoding the heavy and light chain variable regions of the antibodies of the invention, as appropriate, can be derived or produced by any appropriate method, e.g. by cloning or synthesis.

Once nucleic acid fragments encoding the heavy and/or light chain variable regions of the antibodies of the invention have been obtained, these fragments can be further manipulated by standard recombinant DNA techniques, for example to convert the variable region fragments into full length antibody molecules with appropriate constant region domains, or into particular formats of antibody fragment discussed elsewhere herein, e.g. single domain antibodies such as VHH, Fab fragments, scFv fragments, etc., or formats where multiple antibodies (e.g. single domain antibodies/VHH antibodies) are present, e.g. the biparatopic and triparatopic constructs as described herein. Typically, or as part of this further manipulation procedure, the nucleic acid fragments encoding the antibody molecules of the invention are generally incorporated into one or more appropriate expression vectors in order to facilitate production of the antibodies of the invention or for example to facilitate selection or screening, e.g. by incorporating into phage display vectors.

Possible expression vectors include but are not limited to cosmids, plasmids, or modified viruses (e.g. replication defective retroviruses, adenoviruses and adeno-associated viruses), so long as the vector is compatible with the host cell used. Such expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates an expression vector, e.g. a recombinant expression vector containing or comprising a nucleic acid molecule of the invention, or a fragment thereof, and the necessary regulatory sequences for the transcription and translation of the protein sequence encoded by the nucleic acid molecule of the invention. Expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells. For example, the proteins of the invention may be expressed in yeast cells or mammalian cells. In addition, the proteins of the invention may be expressed in prokaryotic cells, such as Escherichia coli.

Other expression vectors would include RNA or mRNA expression vectors, such as for example self-amplifying RNA expression vectors, which can be used to express the antibodies or constructs or combinations of the invention, for example in a subject to be treated.

The proteins of the invention may also be prepared by chemical synthesis using techniques well known in the chemistry of proteins such as solid phase synthesis.

A yet further aspect provides an expression construct or expression vector or expression system (e.g. a viral or bacterial or other expression construct, vector or system) comprising one or more of the nucleic acid fragments or segments or molecules of the invention. Preferably the expression constructs or vectors or systems are recombinant. Preferably said constructs or vectors or systems further comprise the necessary regulatory sequences for the transcription and/or translation of the protein sequence encoded by the nucleic acid molecule of the invention. Preferred constructs etc., are those which allow prolonged or sustained expression of the antibodies (or binding proteins) of the invention within the host target species, e.g. within pigs. Such expression can be transient, e.g. episomal, or more permanent, e.g. via genomic integration, providing sufficient levels and length of expression are achieved in order for a therapeutic or biological effect to be observed. Again self-amplifying RNA expression vectors would be an appropriate example.

A yet further aspect provides a host cell (e.g. a mammalian or bacterial or yeast host cell) or virus, or other delivery vehicle (e.g. a lipid based delivery vehicle such as a liposome or lipid nanoparticle), comprising one or more expression constructs or expression vectors of the invention. Also provided are host cells or viruses or delivery vehicles, comprising one or more of the nucleic acid molecules of the invention. A host cell (e.g. a mammalian host cell or bacterial host cell, or yeast host cell) or virus expressing an antibody (or binding protein) or construct or combination, of the invention or a delivery vehicle comprising an expression construct or nucleic acid molecule of the invention forms a yet further aspect.

Such expression constructs or vectors or systems, or host cells or viruses or delivery vehicles, or other nucleic acid products or fragments encoding the antibodies (or binding proteins) or constructs or combinations of the invention can be administered as therapeutic agents to a subject to allow the production of the antibodies (or binding proteins) etc., of the invention in situ within the subject and thereby exert their therapeutic effects.

A yet further aspect of the invention provides a method of producing (or manufacturing) an antibody, binding protein, protein construct or combination of the present invention comprising a step of culturing the host cells of the invention. Preferred methods comprise the steps of (i) culturing, e.g. in a culture medium, a host cell comprising one or more of the recombinant expression vectors or one or more of the nucleic acid sequences of the invention under conditions suitable for the expression of the encoded antibody or binding protein; and optionally (ii) isolating or obtaining the antibody or binding protein from the host cell or from the growth or culture medium/supernatant. Such methods of production (or manufacture) may also comprise a step of purification of the antibody or binding protein product and/or formulating the antibody or product into a composition including at least one additional component, such as a pharmaceutically acceptable carrier or excipient.

In embodiments when the antibody or binding protein of the invention is made up of more than one polypeptide chain (e.g. certain fragments such as Fab fragments or whole antibodies), then all the polypeptides are preferably expressed in the host cell, either from the same or a different expression vector, so that the complete proteins, e.g. antibody proteins of the invention, can assemble in the host cell and be isolated or purified therefrom.

In another aspect, the invention provides a method of binding CD163, comprising contacting a composition comprising CD163 with an antibody of the invention.

In yet another aspect, the invention provides a method of detecting CD163, comprising contacting a composition suspected of containing CD163 with an antibody of the invention, under conditions effective to allow the formation of CD163/antibody complexes and detecting the complexes so formed.

Compositions comprising at least a first antibody (or binding protein) or construct or combination of the invention, or a nucleic acid molecule, expression vector or host cell of the invention, constitute a further aspect of the present invention. Formulations (compositions) comprising one or more antibodies or constructs or combinations of the invention, or a nucleic acid molecule, expression vector or host cell of the invention, in admixture with a suitable diluent, carrier or excipient constitute a preferred embodiment of the present invention. Such formulations may be for pharmaceutical use, e.g. for use in animal health applications or veterinary use, e.g. in farming, and thus compositions of the invention are preferably pharmaceutically acceptable or acceptable for administration to non-human animals, e.g. mammals, preferably pigs. Suitable diluents, excipients and carriers are known to the skilled man.

The compositions according to the invention may be presented, for example, in a form suitable for oral, nasal, parenteral (e.g. intramuscular, subcutaneous or intradermal), intravenous, topical or rectal administration. Intramuscular administration is particularly convenient.

The active compounds (e.g. the antibodies of the invention) as defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Suitable dosage units can be determined by a person skilled in the art.

The pharmaceutical compositions may additionally comprise further active ingredients (e.g. as described elsewhere herein) in the context of co-administration regimens or combined regimens.

A further aspect of the present invention provides the anti-CD163 antibodies (or binding proteins) or constructs or combinations defined herein, or nucleic acid molecules, expression vectors or host cells of the invention, for use in therapy, in particular for use in the treatment or prevention of any disease or condition associated with CD163 or where CD163 has a role, for example a causative (e.g. a wholly or partially causative role) or an essential role. For example, the anti-CD163 antibodies of the invention can be used in the treatment or prevention of any infection caused by a virus or other pathogen, wherein said infection is associated with CD163, or where CD163 has a role, for example a causative (e.g. a wholly or partially causative role), or an essential role. Exemplary diseases are PRRSV infection or Simian Haemorrhagic Fever Virus (SHFV). Put another way, in accordance with the present invention the anti-CD163 antibodies (or binding proteins) or constructs or combinations, etc., may target and inhibit or reduce the function of CD163, in particular CD163 expressed on or in PAMs or other CD163 positive cells. Thus, the anti-CD163 antibodies (or binding proteins) or constructs or combinations, etc., defined herein may be used in the treatment or prevention of any disease or condition where inhibition of CD163 or blockade or reduction of CD163 function is useful.

Preferred embodiments provide the anti-CD163 antibodies (or binding proteins) or constructs or combinations, etc., of the invention for use in the treatment or prevention of infections in pigs, preferably virus infection in pigs. Particularly preferred is the treatment or prevention of PRRSV infection. In embodiments where pigs are treated, the anti-CD163 antibodies (or binding proteins) or constructs or combinations, etc., of the invention are typically anti-porcine CD163 antibodies (or binding proteins), or comprise or encode such anti-porcine CD163 antibodies (or binding proteins).

CD163 is believed to be the likely receptor for all PRRS viral strains. However, as described elsewhere herein, there are two species of PRRSV; PRRSV-1 and PRRSV-2. Although the PRRSV-1 and PRRSV-2 viruses are phenotypically similar at several levels, there are differences in the viral species. The antibodies (or binding proteins) or constructs or combinations, etc., of the invention can be used to treat or prevent PRRSV-1 and/or PRRSV-2 infection, for example PRRSV-1 and PRRSV-2 infection, or to treat or prevent (e.g. specifically treat or prevent) PRRSV-2 infection.

The administration of the binding proteins or antibodies or constructs or combinations, etc., in the therapeutic methods and uses of the invention is carried out in pharmaceutically, therapeutically, or physiologically effective amounts, to subjects (animals, or mammals, e.g. pigs) in need of treatment. Thus, said methods and uses may involve the additional step of identifying a subject in need of treatment.

Treatment of diseases or conditions in accordance with the present invention (for example treatment of pre-existing disease) includes cure of said disease or condition, or any reduction or alleviation of disease (e.g. reduction in disease severity) or symptoms of disease.

The therapeutic methods and uses of the present invention are suitable for prevention of diseases as well as active treatment of diseases (for example treatment of pre-existing disease). Thus, prophylactic and metaphylactic (treating in the face of a disease outbreak, for example treating a group of subjects after the diagnosis of infection and/or clinical disease in part of the group, with the aim of preventing the spread of infectious disease to animals in close contact and/or at significant risk) treatment is also encompassed by the invention. For this reason, in the methods and uses of the present invention, treatment also includes prophylaxis, metaphylaxis or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Preferred subjects for treatment are subjects that have, or are at risk of, PRRSV infection. As described elsewhere herein, the therapeutic methods of the invention can however be carried out on subjects that have other (non-PRRSV) infections, e.g. have other bacterial or viral infections, or even complex (multi-pathogen) infections. In particular such therapeutic methods are suitable for treating subjects that contain measurable levels of soluble CD163 in their serum, or soluble CD163 levels that are elevated, e.g. significantly elevated, over levels found in healthy subjects.

Clinical symptoms of for example PRRSV infection include foetal reabsorption, still-births and late-term abortion in pregnant sows or gilts, and respiratory diseases and syndromes, e.g. respiratory distress, in all pigs, and especially young pigs and piglets. Other symptoms include lack of appetite (which often leads to decreased growth rates or reduced body weights, or reduced body weight gain, e.g. reduced average daily body weight gain), fever, lethargy, respiratory distress (e.g. pneumonia or pneumonia/lung lesions), reproductive failure and diarrhoea (especially in young piglets), Central Nervous System (CNS) signs and death. Subjects with PRRSV infection also have susceptibility to endemic diseases such as meningitis, Glassers disease, exudative dermatitis, sarcoptic mange and bacterial bronchopneumonia is commonly reported as increasing (Diseases of Swine, Eleventh Edition, Editor(s): Jeffrey J. Zimmerman Locke A. Karriker Alejandro Ramirez Kent J. Schwartz Gregory W. Stevenson Jianqiang Zhang. First published: 29 Mar. 2019), or infection with PCV2. Many of such diseases are generally managed by the use of antimicrobial products such as antibiotics. Consequently, the invention has a role in the reduction of antimicrobial product use on-farm.

Thus, the antibodies or binding proteins or constructs or combinations of the invention can be used to treat or prevent clinical disease or symptoms, e.g. clinical disease or symptoms associated with PRRSV infection or downstream endemic diseases such as those outlined above, or to reduce virus, e.g. PRRSV, circulation (e.g. viral load in serum) or to prevent infection (e.g. first infection) or new infection (e.g. second or subsequent infection), e.g. PRRSV infection (e.g. first PRRSV infection) or new PRRSV infection (e.g. second or subsequent PRRSV infection).

Preferred subjects for treatment in accordance with the present invention thus include all types of pigs (also sometimes referred to as swine), for example any pig, swine, or porcine species, including pigs of all ages and species providing they are susceptible to or are capable of being infected with pathogens as defined herein, and in particular PRRSV. Piglets, especially young piglets or live-born piglets from infected sows (up to 80% of which will die), are particularly preferred subjects, as are nursery pigs (post-weaned pigs that are for example up to 12 weeks old), and growing or fattening pigs (e.g. pigs up to the age of slaughter), in particular growing pigs. Pre-weaned piglets, e.g. piglets up to 4 weeks old (especially those of infected sows, where the infection may be transmitted via the mammary gland secretions of an infected sow) are also preferred subjects to be treated, as are gilts, sows and pregnant sows. As preferred subjects include all types of pigs, it should be understood that the subjects for treatment in all embodiments and aspects include groups of subjects, e.g. herds and litters, but the treatment of individual animals is also not excluded.

In some embodiments, e.g. where prevention is concerned, the subject is a subject at risk of being affected by the disease or condition in question, for example at risk of being infected with a pathogen or virus (e.g. PRRSV) as described above and developing disease. Such a subject may be a healthy subject or a subject not displaying any symptoms of disease or any other appropriate “at risk” subject. In another embodiment the subject is a subject having, or suspected of having (or developing), or potentially having (or developing) the disease or condition in question as described above.

Alternatively viewed, the present invention provides a method of treating or preventing a disease or condition associated with CD163 or where CD163 has a role, for example a causative (e.g. a wholly or partially causative role) or an essential role, which method comprises administering to a subject in need thereof a therapeutically effective amount of an anti-CD163 antibody (or binding protein) or construct or combination of the invention as defined herein. Appropriate diseases or conditions or subjects are described elsewhere herein.

The treatment or prevention of infections in pigs, preferably virus infection in pigs is preferred. Particularly preferred is the treatment or prevention of PRRSV infection, e.g. to treat or prevent PRRSV-1 and/or PRRSV-2 infection, for example PRRSV-1 and PRRSV-2 infection, or to treat or prevent (e.g. specifically treat or prevent) PRRSV-2 infection.

Thus, a yet further aspect provides a method of treatment or prevention of PRRSV infection in a pig, e.g. treatment or prevention of PRRSV-1 and/or PRRSV-2 infection in a pig, which method comprises administering to a subject in need thereof a therapeutically effective amount of a monoclonal antibody which binds to porcine CD163. Appropriate CD163 antibodies (or binding proteins) or constructs or combinations for use in such methods are described herein.

Thus, a yet further aspect provides a method of treatment or prevention of PRRSV infection in a subject, preferably a pig, e.g. a method of treatment or prevention of PRRSV-1 and/or PRRSV-2 infection in said subject, which method comprises administering to a subject in need thereof a therapeutically effective amount of the antibody, binding protein or combinations of the invention. Such therapeutic methods may alternatively involve the administration of one or more nucleic acid molecules, expression vectors or host cells of the invention.

A therapeutically effective amount will be determined based on the clinical assessment and can be readily monitored.

Embodiments of the therapeutic uses of the invention described herein apply, mutatis mutandis, to this aspect of the invention.

Further alternatively viewed, the present invention provides the use of an anti-CD163 antibody (or binding protein) or construct or combination of the invention, e.g. a monoclonal antibody of the invention, as defined herein in the manufacture of a medicament for use in therapy. Preferred therapeutic uses are described elsewhere herein, in particular for use in the treatment or prevention of any disease or condition associated with CD163 or where CD163 has a role, for example a causative (e.g. a wholly or partially causative role) or an essential role. For example, the anti-CD163 antibodies (or binding proteins) of the invention can be used in the treatment or prevention of any infection caused by a virus or other pathogen, wherein said infection is associated with CD163, or where CD163 has a role, for example a causative (e.g. a wholly or partially causative role), or an essential role. Put another way, in accordance with the present invention the anti-CD163 antibodies (or binding proteins) may target and inhibit or reduce the function of CD163, in particular CD163 expressed on or in PAMs or other CD163 positive cells. Thus, the anti-CD163 antibodies (or binding proteins) or constructs or combinations defined herein may be used in the treatment or prevention of any disease or condition where inhibition of CD163 or blockade or reduction of CD163 function is useful.

Preferred embodiments provide the use of anti-CD163 antibodies (or binding proteins) of the invention in the manufacture of a medicament for use in the treatment or prevention of infections in pigs, preferably virus infection in pigs. Particularly preferred is the treatment or prevention of PRRSV infection, e.g. to treat or prevent PRRSV-1 and/or PRRSV-2 infection, for example PRRSV-1 and PRRSV-2 infection, or to treat or prevent (e.g. specifically treat or prevent) PRRSV-2 infection.

Thus, a yet further aspect provides the use of a monoclonal antibody which binds to porcine CD163, in the manufacture of a medicament for use in the treatment or prevention of PRRS virus infection, preferably PRRSV-1 and/or PRRSV-2 infection in a pig. Appropriate CD163 antibodies (or binding proteins) or constructs or combinations for such uses are described herein.

Thus, a yet further aspect provides the use of an antibody, binding protein or combination of the invention in the manufacture of a medicament for use in therapy in a subject, preferably for use in the treatment or prevention of PRRSV infection in a subject, e.g. for use in the treatment or prevention of PRRSV-1 and/or PRRSV-2 infection in a subject, preferably a pig. Such uses may alternatively involve the use of one or more nucleic acid molecules, expression vectors or host cells of the invention.

Embodiments of the therapeutic uses of the invention described herein apply, mutatis mutandis, to this aspect of the invention.

In some embodiments, the antibodies (or binding proteins) of the invention can be used in combination. For example, a membrane-specific antibody of the invention (e.g. an antibody as defined in Table A) can be used in combination with one, two or more alternative anti-CD163 antibodies (e.g. with an antibody as defined in Table B (or D) and/or an antibody as defined in Table C (or E)). Preferred combinations of anti-CD163 antibodies are those where the individual anti-CD163 antibodies of the combination bind to different epitopes on the CD163 molecule, e.g. biparatopic (two different epitopes) or triparatopic (three different epitopes) constructs as described herein.

Any combination of the VHH antibodies as shown in Tables A, B (or D), and C (or E), can be used.

Preferred combinations comprise:

The VHH antibody of Table A, with the VHH antibody of Table B (or D).

The VHH antibody of Table A, with the VHH antibody of Table C (or E).

The VHH antibody of Table B (or D), with the VHH antibody of Table C (or E).

The VHH antibody of Table A, with the VHH antibody of Table B (or D), and the VHH antibody of Table C (or E).

In all the above combinations, antibodies with the 3 CDRs as shown in Tables A to E, as appropriate, or sequences substantially homologous thereto, can also be used.

Preferred combinations are those that result in improved or increased, preferably significantly improved or increased, therapeutic efficacy as compared to any of the antibodies of the invention (e.g. VHHs) administered as a sole active agent (monotherapy), or sole antibody, or sole anti-CD163 agent. Other preferred combinations are those where the individual anti-CD163 antibodies of the combination bind to different epitopes on the CD163 molecule. As described elsewhere herein, in some embodiments of the invention, the antibodies (or binding proteins) of the invention can also be used in combination with an entity that can extend half life, for example a further antibody, e.g. a VHH antibody, that can extend half life, or any other appropriate half life extending entity.

For such combination treatments using two or more antibodies (or binding proteins) of the invention, the second (or subsequent, e.g. third) anti-CD163 antibody may be administered to a subject substantially simultaneously with the first anti-CD163 antibody of the invention, such as from a single pharmaceutical composition or from two pharmaceutical compositions administered closely together (at the same or a similar time). Alternatively, the second (or subsequent, e.g. third) anti-CD163 antibody of the invention may be administered to a subject at a time prior to or sequential to the administration of the first anti-CD163 antibody of the invention. “At a time prior to or sequential to”, as used herein, means “staggered”, such that the second antibody is administered to a subject at a time distinct to the administration of the first anti-CD163 antibody component. Generally, the two (or more) components may be administered at times effectively spaced apart or together to allow the individual components to exert their respective therapeutic effects, i.e., they are administered in “biologically effective amounts” at “biologically effective time intervals” and are administered as part of the same therapeutic regimen.

As described elsewhere herein, preferably, combinations of anti-CD163 antibodies (or binding proteins) of the invention (and optionally an entity which extends half life), can, if appropriate, conveniently be administered as part of the same molecule or construct, e.g. can be conjugated or linked together, e.g. with an artificial linker. This mode of administration can be particularly appropriate for VHH antibodies (or other types of antibody molecule which are composed of a single polypeptide chain), individual antibodies of which can conveniently be connected by appropriate peptide (or other) linkers, e.g. non-native peptide or artificial linkers, in a single polypeptide chain containing multiple VHH (or other) antibodies, either of the invention or in combination with other VHHs or other antibodies.

Appropriate linkers are well known and described in the art. Exemplary linkers can include GS linkers such as one or more repeats of the G₄S linker (GGGGS, SEQ ID NO:43). The linker used in the constructs used in the attached Examples has the sequence GGGGSGGGGS (SEQ ID NO: 44), i.e. 2 repeats of GGGGS. Linkers with 5 repeats are also used. However, it will be appreciated that linkers (spacers) with other sequences and lengths, e.g. other GS linkers, or other appropriate linkers can also be used.

In such embodiments, antibodies are generally linked together using appropriate techniques, e.g. spacing, such that each component can exert their respective effects, for example binding to CD163. For example, in embodiments where anti-CD163 antibodies of the invention bind to different epitopes on CD163, then combinations of such antibodies are preferred and the constructs are designed appropriately so that each individual antibody can bind to CD163, e.g. to its CD163 epitope.

Thus, in some embodiments the anti-CD163 antibodies (or binding proteins) of the invention may be used as the sole active agent in a treatment regimen (monotherapy), or more than one of anti-CD163 antibodies of the invention can be used in combination, for example as described above. In some embodiments the anti-CD163 antibodies (or binding proteins) of the invention (or combination as appropriate) may be used as the sole active anti-CD163 agent(s) or the sole active anti-CD163 antibodies in a treatment regimen, or they may be the sole active anti-PRRSV agent(s) in a treatment regimen. However, in some embodiments, additional anti-CD163 agents or anti-PRRSV agents can be used.

Thus, the anti-CD163 binding proteins or antibodies of the invention (or combination as appropriate) can be combined with one or more further (additional CD163 targeting or non-CD163 targeting) active agents, e.g. with at least a second therapeutic or biological agent, where the anti-CD163 binding protein or antibody of the invention (or combination of such binding proteins or antibodies), is the first.

The anti-CD163 antibodies (or binding proteins) of the invention (or combination as appropriate) can for example be combined with any other therapeutic agent or vaccine which is useful to treat or prevent the disease in question as described elsewhere herein, for example PRRSV, or another disease.

Appropriate and exemplary administration regimes for such combination treatments can be as described elsewhere herein for combinations of anti-CD163 antibodies.

The invention further includes kits comprising one or more of the antibodies, or constructs or compositions of the invention, or one or more of the nucleic acid molecules encoding the antibodies or constructs of the invention, or one or more recombinant expression vectors comprising the nucleic acid sequences of the invention, or one or more host cells or viruses comprising the recombinant expression vectors or nucleic acid sequences of the invention. Preferably said kits are for use in the methods and uses as described herein, e.g. the therapeutic methods as described herein. Preferably said kits comprise instructions for use of the kit components. Preferably said kits are for treating diseases or conditions as described elsewhere herein, and optionally comprise instructions for use of the kit components to treat such diseases or conditions. Equivalent embodiments with binding proteins of the invention are also provided.

The antibodies (or binding proteins) of the invention as defined herein may also be used as molecular tools for in vitro or in vivo applications and assays. As the antibodies (and binding proteins) have an antigen binding site, these can function as members of specific binding pairs and these molecules can be used in any assay where the particular binding pair member is required.

Thus, yet further aspects of the invention provide a reagent that comprises an antibody (or binding proteins) of the invention as defined herein and the use of such antibodies (or binding proteins) as molecular tools, for example in in vitro or in vivo assays.

The term “decrease” or “reduce” (or equivalent terms) as described herein includes any measurable decrease or reduction when compared with an appropriate control. Appropriate controls would readily be identified by a person skilled in the art and might include non-treated or placebo treated subjects or healthy subjects, or samples or assays where no antibody (or binding protein) of the invention is present. Preferably the decrease or reduction will be significant, for example clinically or statistically significant.

The term “increase” (or equivalent terms) as described herein includes any measurable increase or elevation when compared with an appropriate control. Appropriate controls would readily be identified by a person skilled in the art and might include non-treated or placebo treated subjects or healthy subjects, or samples or assays where no antibody (or binding protein) of the invention is present. Preferably the increase will be significant, for example clinically or statistically significant.

Preferably such increases (and indeed other increases, improvements or positive effects as mentioned elsewhere herein) or such decreases (and indeed other decreases, reductions or negative effects as mentioned elsewhere herein) are measurable increases, decreases, etc., (as appropriate), more preferably they are significant increases, decreases, etc., preferably clinically significant or statistically significant increases, decreases, etc., for example with a probability value of $0.05 or <0.05, when compared to an appropriate control level or value (e.g. compared to an untreated or placebo treated subject or compared to a healthy or normal subject, or the same subject before treatment, or a sample or assay where no antibody (or binding protein) of the invention is present).

Methods of determining the statistical significance of differences between test groups of subjects or differences in levels of a particular parameter are well known and documented in the art. For example herein a decrease or increase in level of a particular parameter or a difference between test groups of subjects is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of ≤0.05 or <0.05.

Tables of Amino Acid Sequences Disclosed Herein and their Sequence Identifiers (SEQ ID NOs)

All amino acid sequences are recited herein from the N-terminus to the C-terminus in line with convention in this technical field.

TABLE A
Clone 39-H17B11

| SEQ ID NO: | Description | Sequence |
|---|---|---|
| 1 | VHH (aa) | QVQLVESGGGLVQAGGSLRLSCAASGRTFSSYAMGWFR |
| | | QTPGKDREFVARIGWTGGTTIYADSVKGRFTISRDNAKNT |
| | | VYLQMNSLKPEDTAVYYCAADQAGWRTAGVRNTYEYDY |
| | | WGQGTQVTVSS |
| 2 | Heavy CDR1 | GRTFSSYA |
| 3 | Heavy CDR2 | IGWTGGTT |
| 4 | Heavy CDR3 | AADQAGWRTAGVRNTYEYDY |
| 5 | Heavy FR1 | QVQLVESGGGLVQAGGSLRLSCAAS |
| 6 | Heavy FR2 | MGWFRQTPGKDREFVAR |
| 7 | Heavy FR3 | IYADSVKGRFTISRDNAKNTVYLQMNSLKPEDTAVYYC |
| 8 | Heavy FR4 | WGQGTQVTVSS |

TABLE B
Clone 19-H03E11

| | | |
|---|---|---|
| SEQ ID NO: | Description | Sequence |

VHCDR3 47(#19); VHH 019 (3E11)

| | | |
|---|---|---|
| 9 | VHH (aa) | QVQLQESGGGLVQVGSSLRLSCVTSGRTPSRYVMGWF |
| | | RQAPGQEREFVAAISWSGRAPYADSVKGRFTISRDNAKN |
| | | TVYLQMNSLKPEDTGVYYCAGGEGAIKWTTLDAYDYWG |
| | | QGTQVTVSS |
| 10 | Heavy CDR1 | RYVMG |
| 11 | Heavy CDR2 | AISWSGRAPYADSVKG |
| 12 | Heavy CDR3 | GEGAIKWTTLDAYDY |
| 13 | Heavy FR1 | QVQLQESGGGLVQVGSSLRLSCVTSGRTPS |
| 14 | Heavy FR2 | WFRQAPGQEREFVA |
| 15 | Heavy FR3 | RFTISRDNAKNTVYLQMNSLKPEDTGVYYCAG |
| 16 | Heavy FR4 | WGQGTQVTVSS |

TABLE C
Clone 17-H03D03

| | | |
|---|---|---|
| SEQ ID NO: | Description | Sequence |

VHCDR3 29(#17); VHH 017 (3D03)

| | | |
|---|---|---|
| 17 | VHH (aa) | QLQLVESGGGLVQPGGSLRLSCAASGFTLDDYTIGWFRQ |
| | | APGKEREGVSCINSITSNTYYADSVKGRFTISRDNAKNTV |
| | | YLQMNSLTAEDTAIYYCAADSGLFSGSSCLKYRAMRFGS |
| | | WGQGTQVTVSS |
| 18 | Heavy CDR1 | DYTIG |
| 19 | Heavy CDR2 | CINSITSNTYYADSVKG |
| 20 | Heavy CDR3 | DSGLFSGSSQLKYRAMRFGS |
| 21 | Heavy FR1 | QLQLVESGGGLVQPGGSLRLSCAASGFTLD |
| 22 | Heavy FR2 | WFRQAPGKEREGVS |
| 23 | Heavy FR3 | RFTISRDNAKNTVYLQMNSLTAEDTAIYYCAA |
| 24 | Heavy FR4 | WGQGTQVTVSS |

TABLE D
Clone 19-H03E11-alternative

| | | |
|---|---|---|
| SEQ ID NO: | Description | Sequence |

VHCDR3 47(#19); VHH 019 (3E11)

| | | |
|---|---|---|
| 9 or 25 | VHH (aa) | QVQLQESGGGLVQVGSSLRLSCVTSGRTPSRYVMGWF |
| | | RQAPGQEREFVAAISWSGRAPYADSVKGRFTISRDNAKN |
| | | TVYLQMNSLKPEDTGVYYCAGGEGAIKWTTLDAYDYWG |
| | | QGTQVTVSS |
| 26 | Heavy CDR1 | GRTPSRYV |
| 27 | Heavy CDR2 | ISWSGRA |
| 28 | Heavy CDR3 | AGGEGAIKWTTLDAYDY |
| 29 | Heavy FR1 | QVQLQESGGGLVQVGSSLRLSCVTS |
| 30 | Heavy FR2 | MGWFRQAPGQEREFVAA |
| 31 | Heavy FR3 | PYADSVKGRFTISRDNAKNTVYLQMNSLKPEDTGVYYC |
| 32 | Heavy FR4 | WGQGTQVTVSS |

TABLE E
Clone 17-H03D03-alternative

| | | |
|---|---|---|
| SEQ ID NO: | Description | Sequence |

VHCDR3 29(#17); VHH 017 (3D03)

| | | |
|---|---|---|
| 17 or 33 | VHH (aa) | QLQLVESGGGLVQPGGSLRLSCAASGFTLDDYTIGWFRQ |
| | | APGKEREGVSCINSITSNTYYADSVKGRFTISRDNAKNTV |
| | | YLQMNSLTAEDTAIYYCAADSGLFSGSSCLKYRAMRFGS |
| | | WGQGTQVTVSS |
| 34 | Heavy CDR1 | GFTLDDYT |
| 35 | Heavy CDR2 | INSITSNT |
| 36 | Heavy CDR3 | AADSGLFSGSSCLKYRAMRFGS |
| 37 | Heavy FR1 | QLQLVESGGGLVQPGGSLRLSCAAS |
| 38 | Heavy FR2 | IGWFRQAPGKEREGVSC |
| 39 | Heavy FR3 | YYADSVKGRFTISRDNAKNTVYLQMNSLTAEDTAIYYC |
| 40 | Heavy FR4 | WGQGTQVTVSS |

The invention will now be further described in the following non-limiting Examples with reference to the following drawings:

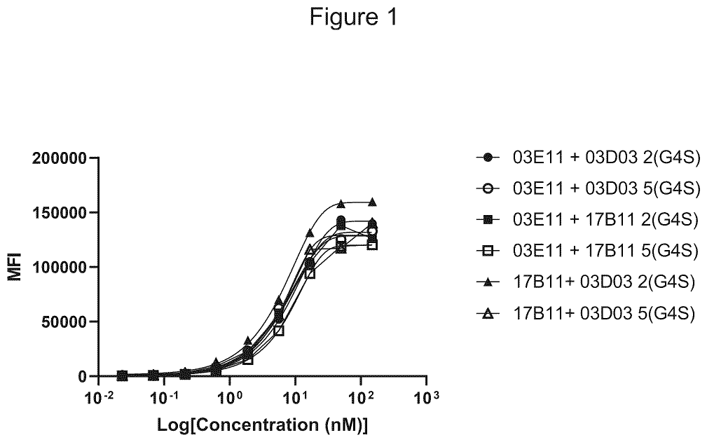

FIG. 1—Dose-response FACS binding assay on pPAM WT cells with example biparatopic antibody candidates.

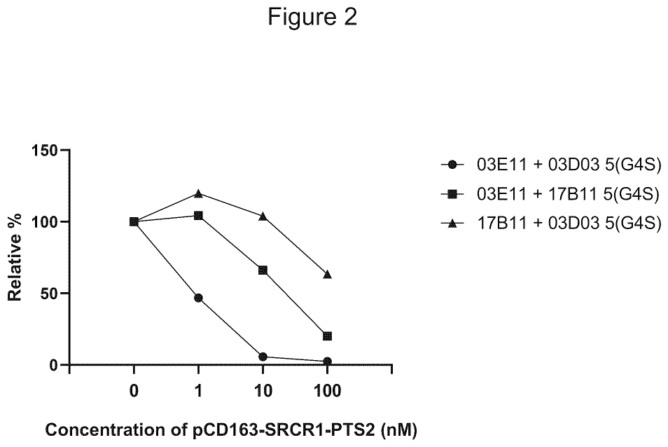

FIG. 2—FACS competition assay on pPAM WT cells with example biparatopic antibody candidates in the presence of increasing concentrations of soluble CD163 protein.

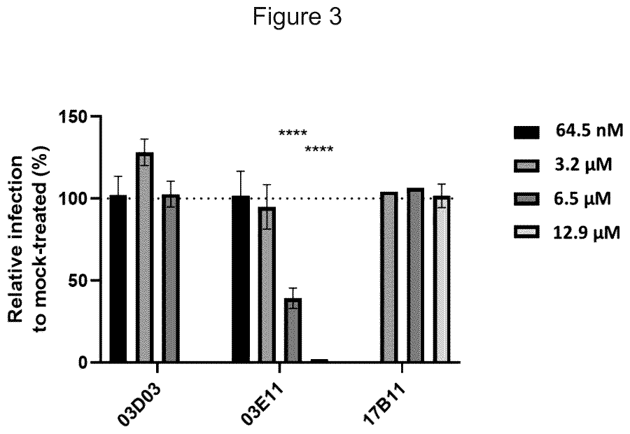

FIG. 3—Infection assay: Monomeric VHHs, infection with PRRSV-1 BOR57.

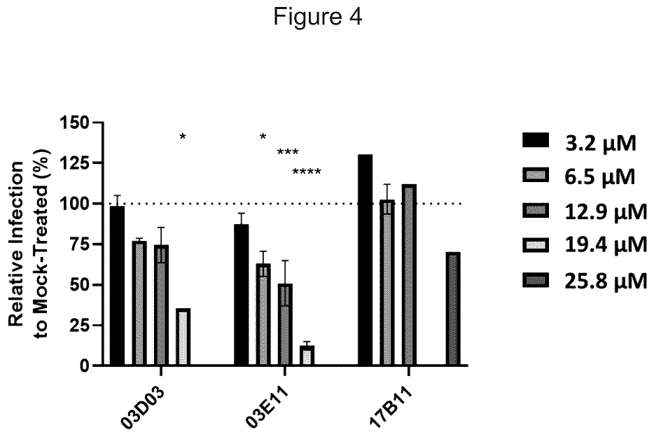

FIG. 4—Infection assay: Monomeric VHHs, infection with PRRSV-2 MN184.

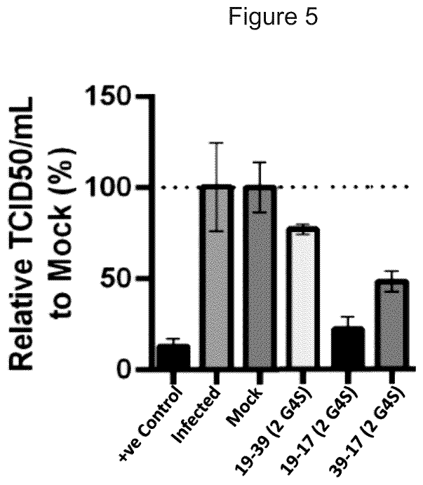

FIG. 5—Infection assay: Biparatopic VHHs, infection with PRRSV-1 BOR57.

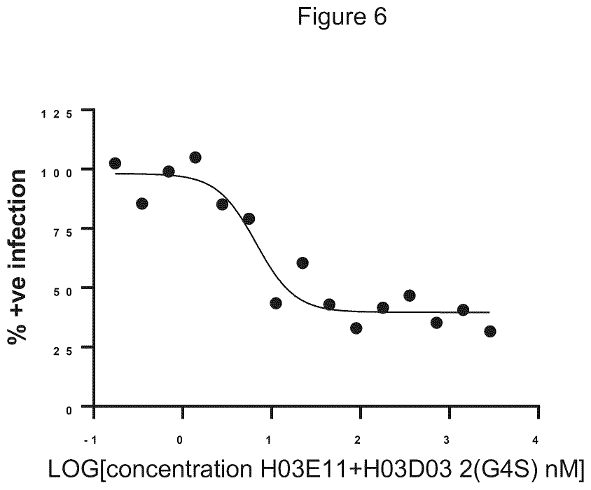

FIG. 6—Infection assay: Biparatopic 03E11+03D03 2(G₄S) dose-response, PRRSV-1 LT3, Sigma RPMI, 10% FBS.

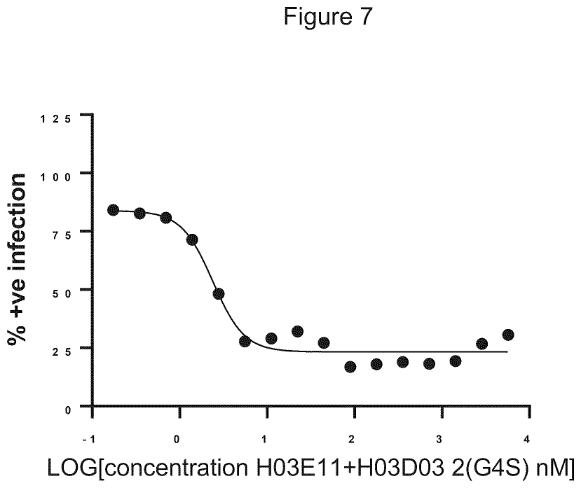

FIG. 7—Infection assay: Biparatopic 03E11+03D03 2(G₄S) dose-response, PRRSV-1 LT3, Sigma RPMI 80% low CD163 porcine serum.

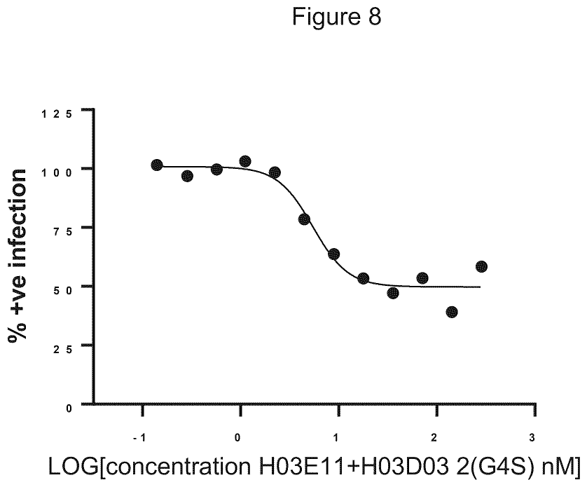

FIG. 8—Infection assay: Biparatopic 03E11+03D03 2(G₄S) dose-response, PRRSV-1 LT3, Sigma RPMI 80% high CD163 porcine serum.

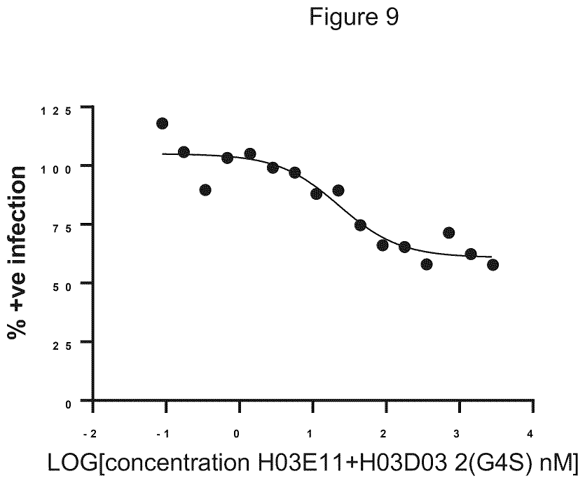

FIG. 9—Infection assay: Biparatopic 03E11+03D03 2(G₄S) dose-response, PRRSV-2 MN184, Sigma RPMI 10% FBS.

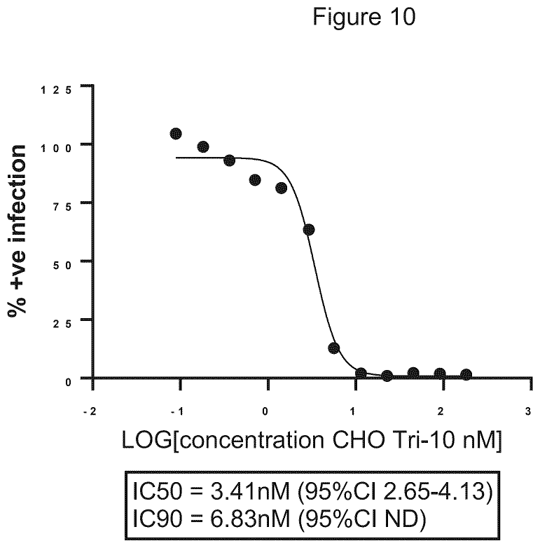

FIG. 10—Infection assay: Triparatopic-10 17B11-03E11-03D03 2(G₄S) dose-response, PRRSV-1 LT3, Sigma RPMI 80% low CD163 porcine serum.

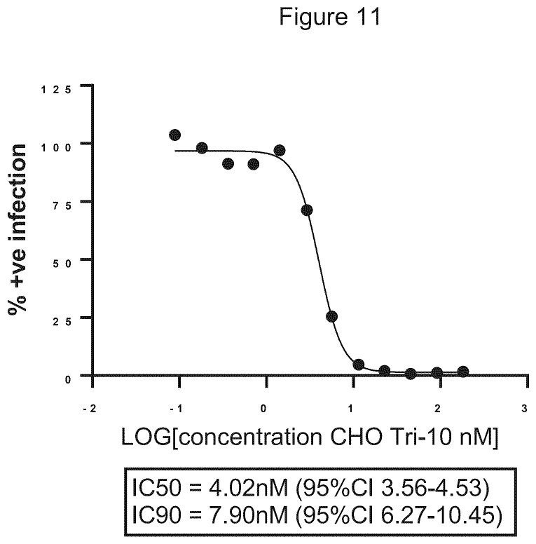

FIG. 11—Infection assay: Triparatopic-10 17B11-03E11-03D03 2(G₄S) dose-response, PRRSV-1 LT3, Sigma RPMI 80% high CD163 porcine serum.

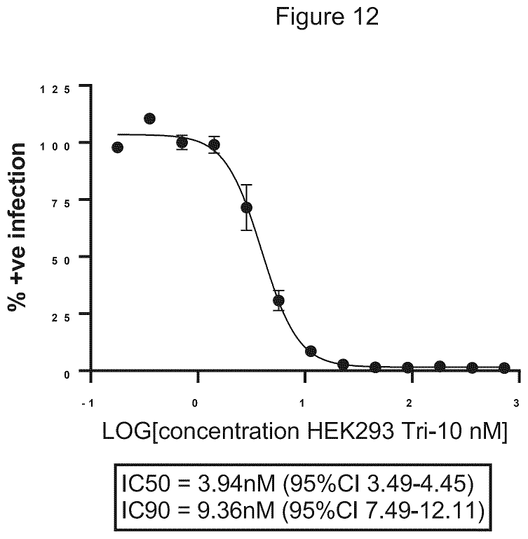

FIG. 12—Infection assay: Triparatopic-10 17B11-03E11-03D03 2(G₄S) dose-response, PRRSV-2 NA174, Sigma RPMI 80% low CD163 porcine serum.

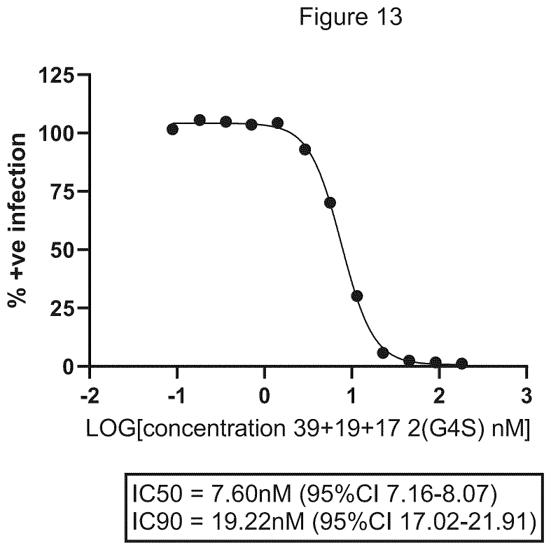

FIG. 13—Infection assay: Triparatopic-10 17B11-03E11-03D03 2(G₄S) dose-response, PRRSV-2 NA174, Sigma RPMI 80% high CD163 porcine serum.

### EXAMPLES

### Example 1: Immunization, Library Generation, Screening and Clone Selection

### Materials and Methods

### Immunization

Single domain antibodies were obtained from llamas immunized with HEK293T cells expressing different porcine CD163 constructs, followed by a final boost with porcine (p) pulmonary alveolar macrophages (pPAM) WT cells. Llamas were injected and given three boosts with HEK293T cells expressing pCD163-SRCR-FL-PST2 (i.e. a full length construct containing from start of SRCR1 to the end of PST2), followed by one final boost with pPAM WT cells. Animals were immunized with 5 injections at two-weekly intervals. Six days after the last boost, sera were collected to define antibody titers against pCD163-SRCR-FL-PST2-His proteins by ELISA. In this ELISA, 96-well plates (Maxisorp; Nunc) were coated with the recombinant proteins. After blocking and adding diluted sera samples, the presence of anti pCD163 antibodies was demonstrated by using Mouse anti-llama IgG (FJB, Cat. nr. FJ1203MAB01B09) followed by Donkey anti-mouse IgG-HRP antibody (JIR, Cat. nr. 715-035-150).

### Library Construction

RNA was extracted from PBMC of 2 immunized llamas (400 ml each). 40 μg of RNA was used for cDNA synthesis using random primers. The cDNA was used in a primary PCR amplification using non-tagged primers annealing at the Leader sequence and Hinge CH1 regions, followed by a secondary PCR amplification introducing restriction endonuclease sites for cloning of VHH genes in pDCL1 phagemid vector. The libraries were electroporated into TG1 E. coli cells and bacterial glycerol stock of the immune libraries were stored at −80° C.

### Selections

Phage production from the llama VHH library pool were used in two consecutive rounds of phage display selection using pCD163 recombinant protein, or HEK293T cells expressing different porcine CD163 constructs, or pPAM WT cells. The first round of selection was carried out on pPAM WT cells with a prior negative counterselection against empty HEK293T WT cells. The second round of selection was carried out on HEK293T pCD163-SRCR-FL-PST2 expressing cells with a prior negative counterselection using pPAMΔ5 domain cells (cells with deletion of SRCR domain 5, Burkhard et al., 2017).

Selections on HEK293T cells expressing pCD163-SRCR-FL-PST2 constructs were performed in PBS buffer pH7.4 with washing of non-specific phage, followed by specific phage elution with trypsin (total elution). Selection rounds on pPAMs were performed using 5E106 cells at pH7.4 (PBS buffer) with washing of non-specific phage, followed by specific phage elution with trypsin (total elution).

Serial dilutions of the eluted phages were performed and used to infect exponentially growing TG1. Infected TG1 was plated on LBCarb100Glu2% plates and enrichment values calculated over the background (without antigen for selection).

### ELISA Screening

Individual clones from the second round of selection conditions outputs were picked into 96-well Master Plates and tested as Periplasmic Extract (P.E.) for binding to pCD163-SRCR-FL-PST2-His or pCD163-SRCR1-9-huFc (i.e. a construct containing from start of SRCR-1 through to the end of SRCR-9 but with no PST2), or huCD6-pPST2-His proteins at pH7.0 via binding ELISA.

For P.E. binding ELISA, MaxiSorp™ high protein-binding capacity 96 well ELISA plates, were coated with 1 μg/ml of pCD163-SRCR-FL-PST2-His or huCD6-pPST2-His, or pCD163-SRCR1-9-huFc protein, diluted in PBS, overnight at 4° C. The next day, plates were washed 3× with PBS Tween 0.05% (pH 7.4) and blocked for 1 hour at room temperature with 250 μl/well of 4% Marvel/PBS. After blocking, plates were washed 3× with PBS Tween 0.05% (pH7.4) and incubated per well with 20 μl of P.E+80 μl in 1% Marvel/PBS (pH7.4), for 1 hour at room temperature (RT) with shaking. Plates were washed 3× with PBS Tween 0.05% (pH7.4) and incubated with 100 μl of anti-c-Myc antibody (Roche; Cat. nr. 11667203001) followed by secondary antibody DAM-HRP (JIR; Cat. nr. 715-035-150) in 1% Marvel/PBS (pH7.4), for 1 hour at RT with shaking. Plates were washed 3× with PBS Tween 0.05% (pH7.4) and the substrate solution (TMB solution) was added to the plates. Reaction was stopped with H2SO4 and plates read in the plate reader at 450 nm.

### Screening on Cells (FACS):

Periplasmic Extract (P.E) from the selected clones were incubated with anti-c-myc antibody (Roche; Cat. nr. 11667203001), specific to the c-myc tag present in the soluble VHH, for 30 minutes with agitation at room temperature (RT). The mix (P.E+anti-c-myc antibody) was added to the pPAM WT or pPAMΔ5 domain (cells with deletion of SRCR domain 5) and incubated for 60 min at 4° C. with gentle shaking.

Cells were washed 3× with 150 μl/well of FACS Buffer and incubated with 50 μl/well of the secondary antibody GAM-APC for 30 min at 4° C. and protected from the light, with shaking.

Cells were washed 3× with 150 μl/well of FACS Buffer and resuspended in 75 μl/well of FACS buffer to be measured in FACS machine (Attune™ N×T) in RL-1 channel (APC channel), and a total of 10000 cells were acquired per sample.

### Sequencing

The positive binders were sent to be sequenced. Clones were classified by families according to the different HCDR3 sequence.

### Expression and Purification of VHH Candidate Antibodies

The synthetic genes codifying to the VHH variable domains with FLAG and His tags were obtained. Each DNA construct was restriction enzyme digested, the insert was gel purified, and each variable domain insert was ligated with a mammalian expression vector pcDNA3.1. ExpiCHO-S cells were transfected with VHH sequences using 40 μg of total DNA plasmid constructs. A 25 mL total volume of cells was used for 8 days of protein production (32° C., 5% CO2). Produced VHH antibodies were captured from clarified supernatants using a HisTrap HP 5 mL IMAC column (GE Healthcare, Cat. nr. 17-5248-02) on an ÄKTA Pure 25 FPLC system. Eluted antibody peak fractions were buffer exchanged to 1×PBS pH 7.4 and concentrated using 3 kDa MCO spin concentrators (Amicon, Cat. nr. UFC900324). Purified protein was analysed by analytical size exclusion chromatography (aSEC) and SDS-PAGE for the presence of correct chains.

Dose-Response ELISA with Purified VHH

MaxiSorp™ high protein-binding capacity 96 well ELISA plates, were coated with 1 μg/ml of pCD163-SRCR1-9-huFc diluted in PBS, overnight at 4° C. The next day, plates were washed 3× with PBS Tween 0.05% (pH 7.4) and blocked for 1 hour at room temperature with 250 μl/well of 4% Marvel. After blocking, plates were washed 3× with PBS Tween 0.05% (pH7.4).

VHH in PBS (pH7.4) were diluted from 200 to 0.0034 nM in 3-fold steps and added to the pCD163-SRCR1-9-huFc coated and blocked ELISA wells for 1 hr at RT.

Plates were washed 3× with PBS Tween 0.05% (pH7.4) and incubated with anti-Histidine-HRP (Miltenyi Biotec, Cat. nr. 130-092-783) in PBS (pH7.4), for 1 hour at RT. Plates were washed 3× with PBS Tween 0.05% (pH7.4) and the substrate solution (TMB solution) was added to the plates. The reaction was stopped with H₂SO₄ and plates read in the plate reader at 450 nm.

### Affinity Determination of Purified VHH by Biacore

To assess the affinity of selected purified clones to pCD163 protein, pCD163-SRCR1-9-huFc and pCD163-1-PST2-His (also referred to herein as pCD163-SRCR-FL-PST2-His) proteins were coated by amine coupling on a CM5 sensor ship (GE Healthcare).

Surface plasmon resonance (SPR) (Biacore 3000, GE Healthcare) was used to determine the binding kinetics of selected single domain antibodies at pH7.4. Approximately 2075 to 2423 RUs of pCD163-SRCR1-9-huFc or 2859 to 3286 RUs of pCD163-1-PST2-His. respectively, at 20 or 30 μg/ml in Acetate buffer pH 5.0 or pH 5.5 were immobilized onto a CM5 chip using the standard amine coupling procedure.

QC of the immobilization was done using a commercial antibody anti-porcine CD163 (BioRad, Cat. nr. MCA2311GA) at 30 nM diluted in HBS-EP pH 7.4 buffer

1×HBS-EP pH 7.4, was utilized as a running buffer during binding kinetic measurements. Purified VHH were injected at 2-fold dilution from 200 nM down to 12.5 nM in HBS-EP pH 7.4, at 30 μl/min for 2 minutes, followed by an off-rate wash for 1 min between injections. Off-rate wash was 300s after the last concentration injection in each cycle. RU levels were restored to base levels after regeneration with two injections of 10 μl of 1 M NaCl, 1 mM Glycine pH 1.5 between samples.

Fitting 1:1 binding with mass transfer was applied to the set of sample curves using the simultaneous fitting option of the BIAevaluation software to calculate the kinetic constants of the antibody-antigen interactions including association rate (ka), dissociation rate (kd) and affinity (KD). Curves were removed from the fitting after visual examination of the residuals and considering the value of Chi2: a minimum of 4 curves were considered for the simultaneous fitting.

### Results & Discussion

Membrane specific binding was confirmed for clones, as exemplified by clone 17B11. As can be seen from Table 1, Table 3, and Table 4, clone 17B11 displayed no binding for the non-cell-surface associated (soluble) CD163 in ELISA, ELISA EC50, or Biacore experiments.

As is seen in Table 2, clone 17B11 does bind to pPAM WT cells, which express CD163. Furthermore, clone 17B11 does not bind to pPAMΔ5 WT cells, indicating that its binding specifically requires the SRCR5 domain of cell-surface expressed CD163.

Collectively these results indicate that clone 17B11 is specific for cell surface associated CD163.

TABLE 1
P.E. ELISA screening results for clone 17B11

P.E. binding ELISA

| | pCD163-SRCR1- | pCD163-1- | huCD6- |
|---|---|---|---|
| | 9-huFc | PST2-His | pPST2-His |
| Clone ID | O.D. 450 nm | O.D. 450 nm | O.D. 450 nm |
| 17B11 | 0.091 | 0.066 | 0.052 |

TABLE 2
P.E. FACS screening results for clone 17B11

| P.E. binding FACS (2nd screening) | |
|---|---|
| pPAM WT | pPAM Δ5 |

| Clone ID | % of binding | MFI | % of binding | MFI |
|---|---|---|---|---|
| 17B11 | 45.00 | 823 | 1.15 | 270 |

TABLE 3
Purified VHH Dose response ELISA for clone 17B11. Binding to pCD163-SRCR1-9-huFc.

| Clone | VHH concentration (nM) | | | | | | | | | | | |
|---|---|---|---|---|---|---|---|---|---|---|---|---|
| ID | 200 | 66.67 | 22.22 | 7.41 | 2.47 | 0.82 | 0.27 | 0.09 | 0.030 | 0.010 | 0.0034 | 0.00 |
| 17B11 | 0.101 | 0.058 | 0.050 | 0.048 | 10.048 | 0.050 | 0.048 | 0.048 | 0.049 | 0.048 | 0.054 | 0.048 |

TABLE 4
Affinity determination by Biacore of clone 17B11

| | pCD163-SRCR1-9-huFc | | | | | pCD163-SRCR1-PST2 | | | | |
|---|---|---|---|---|---|---|---|---|---|---|
| | Kinetics | | | | | Kinetics | | | | |
| Clone | Chi2 | ka | kd | KD | Rmax | Chi2 | | | KD | Rmax |
| ID | (RU2) | (1/Ms) | (1/s) | (nM) | (RU) | (RU2) | ka (1/Ms) | kd (1/s) | (uM) | (RU) |
| 17B11 | N/D | N/D | N/D | N/D | 3.10 | 12.70* | 1.10E+03 | 2.81E−01 | 255 | 13062.30 |

N/D = not able to determine because binding too weak.

The sequence of 17B11 is shown in Table A (it is sometime referred to herein as clone 39)

### Example 2: Functional Assays Using Biparatopic and Triparatopic Constructs Including the 17B11 VHH Antibody

Anti-Porcine CD163 clones 03E11 (H03E11) and 03D03 (H03D03)

Previously selected anti-porcine (p)CD163 VHH clones 03D03 and 03E11 were selected from libraries generated from two llamas immunized with pCD163-SRCR4-7-huFc and a final boost with pCD163-SRCR1-9-huFc. Two consecutive rounds of phage display selection were conducted using pCD163 recombinant protein or pPAM WT cells. Selection rounds on recombinant proteins were performed using 10 μg/ml of pCD163-SRCR1-9-huFc or pCD163-SRCR4-7-huFc pH7.4 (PBS buffer) with washing of non-specific phage, followed by specific phage elution with trypsin (total elution). Selection rounds on pPAM cells were conducted as above.

These clones were identified by P.E. ELISA screening on recombinant protein (soluble CD163) and P.E. FACS screening on cells (membrane CD163). P.E. ELISA screening was on pCD163-SRCR4-7-huFc or pCD163-SRCR5-6-huFc proteins at pH7.0 (PBS). P.E. FACS screening was on pPAM WT and pPAMΔ5 cells. Both these clones showed binding to both soluble porcine CD163 (recombinant protein) and to the membrane form of porcine CD163 (pPAM WT), but did not show significant binding to pPAMΔ5 cells. Thus, these clones are believed to bind to the SRCR5 domain of porcine CD163. The 03E11 antibody has been shown to inhibit either PRRSV-1 or PRRSV-2 infection (see FIGS. 3 and 4) and the sequence of 03E11 is shown in Table B (it is sometime referred to herein as clone 19). The 03D03 antibody has been shown to inhibit PRRSV-2 infection (see FIG. 4) and the sequence of 03D03 is shown in Table C (it is sometime referred to herein as clone 17).

These antibodies were used in conjunction with the 17B11 antibody to construct biparatopic (two antibody) and triparatopic (three antibody) constructs as described below.

### Materials and Methods

### Construction of Biparatopic and Triparatopic Candidate Antibodies

Individual VHH were assembled into biparatopic and triparatopic combinations using flexible linkers of either 2×(G₄S) or 5×(G₄S). These linkers were placed between each of the individual antibodies in the construct. Combinations were made semi-rationally taking into consideration non-competing epitopes and aiming for high affinity and high potency against both PRRSV1 and PRRSV2 sub-types, as well as for the ability to bind cell surface CD163 and block infection in the presence of competing soluble CD163 protein. This non-interference is deemed important considering high levels of soluble CD163 can be present in the serum of animals in the field, in particular animals suffering from bacterial and/or viral infections such as Lawsonia intracellularis infection, which are quite common, and which could act as a sink for any therapeutic agent that did not prefer cell-surface CD163.

Biparatopic constructs (comprising two different anti-porcine CD163 antibodies that are capable of binding different epitopes on porcine CD163) were produced first and assessed for binding to pPAM WT cells. They were further assayed for binding to pPAM WT cells in the presence of increasing concentrations of soluble CD163 in a competition setup. Examples of the biparatopic constructs that were made are summarised in Table 5 and include 03E11+03D03 2(G₄S); 03E11+03D03 5(G₄S); 03E11+17B11 2(G₄S); 03E11+17B11 5(G₄S); 17B11+03D03 2(G₄S); and 17B11+03D03 5(G₄S). 2(G₄S) means that the individual VHHs are each separated by a G₄S linker with two repeats and 5(G₄S) means that the individual VHHs are each separated by a G₄S linker with five repeats.

Triparatopic constructs (comprising three different anti-porcine CD163 antibodies that are capable of binding different epitopes on porcine CD163) were also produced. The triparatopic constructs that were made are summarised in Table 6 and include 03E11+03D03+17B11 2(G₄S), sometimes referred to as Tri-2; 03E11+17B11+03D03 2(G₄S); 03D03+03E11+17B11 2(G₄S): 03D03+17B11+03E11 2(G₄S): 17B11+03E11+03D03 2(G₄S), sometimes referred to as Tri-10; and 17B11+03D03+03E11 2(G₄S). 2(G₄S) means that the individual VHHs are each separated by a G₄S linker with two repeats.

### Expression and Purification of Biparatopic and Triparatopic Candidate Antibodies

Appropriate combinations of the synthetic genes codifying to the VHH variable domains with FLAG and His tags were ligated into a mammalian expression vector pcDNA3.1 or pcDNA3.4 with appropriate linkers. ExpiCHO-S or HEK293T cells were transfected with the DNA plasmid constructs and cultured for between 7-10 days for protein production (32° C., 5% CO2). Produced biparatopic and triparatopic VHH antibody constructs were captured from clarified supernatants using a HisTrap IMAC column (GE Healthcare, Cat. nr. 17-5248-02) on an FPLC system. Eluted antibody peak fractions were buffer exchanged to 1×PBS pH 7.4 and concentrated using 3 kDa MCO spin concentrators (Amicon, Cat. nr. UFC900324). Purified protein was analysed by analytical size exclusion chromatography (aSEC) and SDS-PAGE for the presence of correct chains.

Dose-Response FACS on pPAM WT Cells

Biparatopic and triparatopic antibody constructs in PBS (pH7.4) were diluted in a 3-fold serial dilutions from 150 nM down to 0.023 nM in FACS buffer (0.5% FBS, 0.5 mM EDTA in 1×PBS pH7.4). Biparatopic and triparatopic candidates were incubated with anti-FLAG-Biotin (Sigma, Cat. nr. F9291), for 30 min on ice with shaking. This mixture was added to pPAM WT cells and incubated for 60 min on ice with gentle shaking. Cells were washed 3×with 150 μl/well of FACS buffer and incubated with 50 μl/well of the secondary detection reagent Streptavidin R-P.E. conjugate antibody (Invitrogen, Cat. nr. SA10044) for 30 min on ice with shaking and protected from light. Cells were then washed 3× with 150 μl/well of FACS Buffer and resuspended in 50 μl/well of FACS buffer to be measured on a FACS machine (Attune™ N×T), and a total of 10000 cells were acquired per sample.

FACS Competition Assay on pPAM WT Cells: Biparatopic Antibody Candidate Binding in Presence of Soluble CD163 Protein

Biparatopic candidates were diluted to 1 nM in FACS buffer and mixed with pCD163-SRCR1-9-PST2 at a final concentration of 0, 1, 10 and 100 nM in FACS buffer. This mixture was then incubated with pPAM WT cells for 60 min on ice, shaking. For VHH detection, the anti-FLAG-Biotin (Sigma, Cat. nr. F9291) antibody was added to the cell mixture, for 30 min on ice with shaking. Cells were washed 3× with 150 μl/well of FACS buffer and incubated with the secondary detection reagent anti-mouse IgG-APC (Invitrogen, Cat. nr. A865) for 30 min on ice with shaking and protected from light. Cells were washed 3× with 150 μl/well of FACS Buffer and resuspended in 50 μl/well of FACS buffer to be measured on a FACS machine (Attune™ N×T), and a total of 10000 cells were acquired per sample.

### Results and Discussion

As can be seen in FIG. 1 and Table 5, biparatopic VHH candidates bound very well to pPAM WT cells. As demonstrated in FIG. 2, biparatopic combinations can have reduced binding to pPAM WT cells in the presence of competing soluble CD163, as exemplified by 03E11+03D03 5(G₄S) that is significantly inhibited at 10 nM and 100 nM of competing soluble CD163. However, the other biparatopics that contain the membrane specific anti-CD163 17B11 VHH antibody (exemplified by 03E11+17B11 5(G₄S) and 17B11+03D03 5(G₄S)) maintain higher levels of binding to pPAM WT cells, even in the presence of a vast excess, up to 100 nM soluble CD163 protein. Thus, the data clearly shows that biparatopic combinations containing 17B11 can maintain good levels of binding to pPAM WT cells in the presence of 10 nM, and even in the presence of up to 100 nM, competing soluble CD163 protein, compared to biparatopic combinations that do not contain 17B11.

TABLE 5
Dose-response FACS on pPAM WT cells with
example biparatopic antibody candidates

| | EC50 (nM) on |
|---|---|
| Sample ID | PAM WT cells |
| 03E11 + 03D03 2(G₄S) | 8.4 |
| 03E11 + 03D03 5(G₄S) | 6.5 |
| 03E11 + 17B11 2(G₄S) | 6.8 |
| 03E11 + 17B11 5(G₄S) | 8.4 |
| 17B11 + 03D03 2(G₄S) | 6.6 |
| 17B11 + 03D03 5(G₄S) | 6.6 |

As can be seen in Table 6, triparatopic VHH candidates also bound very well to pPAM WT cells.

TABLE 6
Dose-response FACS on pPAM WT cells with
example triparatopic antibody candidates

| | EC50 (nM) on |
|---|---|
| Sample ID | PAM WT cells |
| 03E11 + 03D03 + 17B11 2(G₄S) | 9.1 |
| 03E11 + 17B11 + 03D03 2(G₄S) | 18.2 |
| 03D03 + 03E11 + 17B11 2(G₄S) | 3.8 |
| 03D03 + 17B11 + 03E11 2(G₄S) | 13.6 |
| 17B11 + 03E11 + 03D03 2(G₄S) | 11.6 |
| 17B11 + 03D03 + 03E11 2(G₄S) | 8.0 |

### Example 3: Inhibition of Porcine Respiratory and Reproductive (PRRS) Virus Infection of Primary Porcine Alveolar Macrophage Cells by Biparatopics and Triparatopic Constructs Including the 17B11 VHH Antibody

### Materials and Methods

### PRRS Virus Infection Protocol

### Reagents

### Control Antibodies:

Primary antibody: Anti-PRRS 1AC7, Ingenasa
Secondary antibody: Goat anti-mouse IgG (H+L) Alexa Fluor Plus 488, ThermoFisher, A32723

### Culture Medium:

Complete RPMI, 10% FBS, 80% low sCD163 porcine serum, or 80% high sCD163 porcine serum, Ultraglutamine, Pen/Strep (sCD163 is soluble CD163)
PAM Isolation: Porcine Alveolar Macrophage Isolation was conducted as described in Burkard et al., 2017.

### Virus Isolates:

Type 1 Virus: BOR57 isolate (Roslin Institute, Edinburgh, UK)
Type 1 Virus: LT3 (PRRSV1 subtype 2 strain, Roslin Institute, Edinburgh, UK)
Type 2 Virus: MN184 a US strain (Han et al 2006)
Type 2 Virus: NA174 (Roslin Institute, Edinburgh, UK)

### Infection Protocol

### Day 1—Seed Cells

Porcine alveolar macrophage cells were seeded at 20 million per plate in complete RPMI in 48-well plate and left in CO2 incubator overnight

### Day 2—VHH Treatment & Infection Challenge

1. Pre-Treatment (30 minutes before infection)
   a. Aspirate medium off cells
   b. Add 100 μL culture medium to untreated uninfected and untreated infected controls
   c. Add 20 μL PBS in 100 μL culture medium to mock treated infected controls
   d. Add appropriate amount of VHH stock in 100 μL culture medium to treated and infected samples
   e. Return plate to CO₂ incubator for 30 minutes
2. Thaw virus stock and sonicate for 15 seconds before use
3. Infection Challenge (2 hours)
   a. Remove culture medium from cells and reserve VHH-containing culture medium for overnight incubation step
   b. Add 100 μL culture medium to untreated uninfected controls
   c. Add 10 μL virus in 100 μL culture medium to untreated infected controls
   d. Add 10 μL virus plus 20 μL PBS in 100 μL culture medium in mock treated infected controls
   e. Add appropriate amount of VHH stock and 10 μL virus in 100 μL culture medium to treated and infected samples
   f. Gently agitate plate and return to CO₂ incubator
   g. Gently agitate plate every 15 minutes for 2 hours
4. Overnight Incubation (15 hours)
   a. Aspirate medium off cells
   b. Add 100 μL culture medium to untreated uninfected controls and untreated infected controls
   c. Add 20 μL PBS in 100 μL culture medium to mock treated infected controls
   d. Add reserved VHH containing culture medium from the pre-treatment step to appropriate samples
   e. Return plate to CO₂ incubator for 15 hours

### Day 3—Assay and Viral Infection Read-Out

5a. Direct lysis RT-qPCR protocol for quantification of viral RNA in culture supernatant
   a. 5 μl culture supernatant collected at 24 HPI
   b. 1:2 dilution made with lysis buffer (20 mM Trizma hydrochloride buffer pH 7.5, 300 mM NaCl, 2.5% Igepal® CA-630, 1:2000 RNasin® Plus RNase Inhibitor) and mixed
   c. Samples incubated at RT for 20 mins
   d. Samples diluted with nuclease-free H2O (1:5) and used in subsequent qRT-PCR protocol (optimised and validated primers for PRRSV)
   e. Readout: Relative TCID50/ml to mock-treated (%)
5b. In-well Fix and Stain Protocol
   a. Aspirate medium from cells
   b. Fix cells in 4% formaldehyde/PBS++ (with calcium & magnesium) solution at RT for 30 minutes
   c. Wash once with PBS++
   d. Permeabilise with Triton-X (1% in PBS++) at RT for 5 minutes
   e. Wash once with PBS++ or blocking solution (PBS++/5% FBS)
   f. Block with blocking solution (PBS++/5% FBS) at RT for 20 minutes
   g. Add primary antibody Anti-PRRS 1AC7 at 1:5000 to all wells except for unstained controls and secondary antibody only controls
   h. Incubate at RT for 1 hour
   i. Wash three time with PBS++
   j. Add secondary antibody Goat anti-mouse IgG (H+L) Alexa Fluor Plus 488 at 1:5000 to all wells except for unstained controls
   k. Incubate at RT for 45 minutes
   l. Wash three time with PBS++
   m. Add 300 μL PBS++
   n. Scrape cells using a wide bore p200 pipette tip, then scrape around the edges of the well with a normal p200 tip and then use a p1000 to wash the well surface 3× and collect the cells for transfer into a FACs tube.
   o. Measurements were conducted on Fortessa ×20

### Soluble CD163 Containing Serum

Low soluble CD163 containing serum was harvested from a healthy pig. High soluble CD163 serum was harvested from a pig with an ongoing Lawsonia intracellularis infection. Lawsonia intracellular infection leads to a shedding of CD163 and a subsequent high level of soluble CD163 in the serum, while any serum Lawsonia intracellularis would not interfere with the viral PRRS Virus infection assay.

Serum concentrations of soluble CD163 were calculated against a standard serum previously checked by ELISA for concentration. Low CD163 serum from the healthy pig and high CD163 serum from the infected pig were diluted 1:1 and 1:9, respectively, in PBS before concentrations were measured by dot-blot using the primary detection antibody anti-PRRS 1AC7 (Ingenasa), and the secondary antibody goat anti-mouse IgG (H+L) Alexa Fluor Plus 488 (ThermoFisher, A3272)

The healthy pig serum was found to contain 0.4 mg/l (±0.015 STDEV), and infected serum was found to contain 4.5 mg/l (+0.45 STDEV) soluble CD163. Media containing 10% FBS contains no soluble CD163.

### Results and Discussion

The ability of example individual VHH, biparatopic, and triparatopic VHH constructs to inhibit infection of pPAM host cells by PRRS virus family members is described below. The assay, as described above was used to measure the extent of virus infection as quantified by the ability to propagate virus, as measured by FACS or RT-qPCR following a 17 hour infection cycle.

The data clearly show VHH able to inhibit productive infection of porcine alveolar macrophage cells by both PRRSV-1 (see FIG. 3) and PRRSV-2 (see FIG. 4) isotypes. The individual VHH showing activity in the infection assays could be divided into those that were effective against both PRRSV-1 and PRRSV-2 infection (exemplified by 03E11), and those which did not show any inhibitory activity against PRRSV-1 infection, but which showed inhibitory activity against PRRSV-2 infection (exemplified by 17B11 and 03D03).

Biparatopic Combinations of VHH 03D03 (clone 17), 03E11 (clone 19), and 17B11 (clone 39) were used in infection assays using the BOR57 PRRSV-1 (FIG. 5). The best performing biparatopic construct (19-17, 03E11-03D03 2(G₄S)) was then used in infection assays using the LT3 PRRSV-1 (FIGS. 6 to 8), or MN184 PRRSV-2 (FIG. 9) in the presence of medium containing 10% FBS, low soluble CD163 porcine serum, or high soluble CD163 containing serum

Biparatopics which did not contain a membrane specific anti-CD163 VHH, as exemplified by 03E11+03D03 (19-17), were able to reduce the infection potential of PRRSV-1 virus by a maximum of approximately 65% to 75% in media containing only 10% FBS (see FIG. 6). In media containing 80% low CD163 porcine serum such biparatopics were able to reduce the infection potential of PRRSV-1 virus by approximately 75% (see FIG. 7).

The data also show that in the media containing 80% high soluble CD163 porcine serum, however, the infection potential of PRRSV-1 virus was only reduced by approximately 50% (see FIG. 8). The biparatopic 03E11+03D03 was also susceptible to competition from soluble CD163 in binding to pPAM WT cells in the FACS competition assay above (FIG. 2). This clearly demonstrates that competing soluble CD163 can reduce the effectiveness of potential therapeutics that are not specific for membrane associated CD163 and are sensitive to blocking by competing soluble CD163.

Biparatopic combinations, as exemplified by 03D03+03E11, are able to reduce the infection potential of PRRSV-2 virus by approximately 40% (see FIG. 9) in media containing 10% FBS. Although effective, the effect on PRRSV-2 infection was thus lower than on PRRSV-1 infection, even when no soluble CD163 is present. It would be advantageous to improve this efficacy.

Whereas biparatopic combinations such as 03D03+03E11 can display reduced efficacy in the presence of high soluble CD163, the data also show that in the same assays, triparatopic combinations that contain a membrane-specific VHH, such as 17B11, and exemplified by the triparatopics Tri-2 and Tri-10, can reduce the infection potential of PRRSV-1 and PRRSV-2 by 100%, even in the presence of high soluble CD163. Moreover, triparatopic combinations that combine a membrane specific anti-CD163 VHH (e.g. 17B11) with other VHH that individually are able to inhibit infection of PRRSV-2 (e.g. 03D03 or 03E11), and/or PRRSV-1 (e.g. 03E11), are able to combine these properties in advantageous ways.

Triparatopics assembled from VHH that individually are able to block infection of PRRSV-2, or PRRSV-1 and PRRSV-2, as well as a membrane specific anti-CD163 VHH are exemplified by triparatopics Tri-2 (03E11-03D03-17B11 2(G₄S)) and Tri-10 (17B11-03E11-03D03 2(G₄S)).

Such triparatopics are able to completely inhibit the infection potential of PRRSV-1 with very low IC50 values in the range 2.81-4.02 nM in both low soluble CD163 serum (see FIG. 10, and Table 7) and high soluble CD163 serum (see FIG. 11, and Table 7).

In addition, such triparatopics are able to completely inhibit the infection potential of PRRSV-2 with very low IC50 values in the range 2.13-7.60 nM in both low soluble CD163 serum (see FIG. 12, and Table 8) and high soluble CD163 serum (see FIG. 13, and Table 8).

The data clearly shows that combinations of VHH that can individually block either PRRSV-1 and PRRSV-2, or PRRSV-2, with a membrane-specific VHH that shows reduced sensitivity to competing soluble CD163 can result in an excellent blockade of infection by PRRSV-1 or PRRSV-2 family members. Advantageously, such combinations can enhance blockade of infection by PRRS virus family members in the presence of elevated levels of soluble CD163 such as those seen in an ongoing infection where soluble CD163 is present at significant levels in the serum and could act as a sink. Such multiparatopic VHH would have obvious and significant advantages as therapeutic agents for the prevention and treatment of PRRS virus infection.

TABLE 7
Triparatopic PRRSV1 LT3 infection assay summary

| | Low CD163 | | High CD163 | |
|---|---|---|---|---|
| | porcine serum | | porcine serum | |
| Triparatopic clone | IC50 (nM) | IC90 (nM) | IC50 (nM) | IC90 (nM) |
| Tri-2: 03E11-03D03- | 2.81 | 3.83 | 2.96 | 4.44 |
| 17B11 2(G₄S) | | | | |
| Tri-10: 17B11-03E11- | 3.41 | 6.83 | 4.02 | 7.90 |
| 03D03 2(G₄S) | | | | |

TABLE 8
Triparatopic PRRSV2 infection assay summary

| | | | Strain NA174 | | | |
|---|---|---|---|---|---|---|
| | Strain MN184 | | Low CD163 porcine | | High CD163 porcine | |
| | 10% FBS | | serum | | serum | |
| Triparatopic clone | IC50 (nM) | IC90 (nM) | IC50 (nM) | IC90 (nM) | IC50 (nM) | IC90 (nM) |
| Tri-2: 03E11-03D03-17B11 | 2.13 | 2.73 | 4.99 | 9.38 | 5.01 | 8.32 |
| 2(G₄S) | | | | | | |
| Tri-10: 17B11-03E11-03D03 | 2.84 | 3.19 | 3.94 | 9.36 | 7.60 | 19.22 |
| 2(G₄S) | | | | | | |

### REFERENCES

Burkard C., et al Precision engineering for PRRSV resistance in pigs: Macrophages from genome edited pigs lacking CD163 SRCR5 domain are fully resistant to both PRRSV genotypes while maintaining biological function PLoS Pathogens, 13(2) 2017: e1006206
Han et al., 2006, Complete genome analysis of RFLP 184 isolates of porcine reproductive and respiratory syndrome virus, Virus Res., 122: 175-182.

### Example 4: Efficacy of a Repeated Administration of a Triparatopic Construct, Including the 17B11 Antibody, Against an Experimental Infection with PRRSV-1 (Strain LT3) in Piglets

### Materials and Methods

### Animals

26 piglets (Sus scrofa domesticus), healthy and aged approximately 5-6 weeks at challenge.
Breed: Large White×Pietrain,
Males and females,
PCR negative and seronegative to PRRSV-1 and PRRSV-2 before challenge

### Challenge

Challenge strain: LT3 strain of PRRSV-1 at approximately 10⁶ TCID₅₀/mL (Roslin institute, Edinburgh, UK),
Inoculum: 5 mL administered intranasally to each piglet, once on Day 0 (D0) at Time 0 (T0)
   Treatment Group: VHH (Tri2)—12 Pigs Challenged on Day 0 at T0 with LT3
VHH: Triparatopic 03E11+03D03+17B11 2(G₄S), referred to as “Tri2”.
Formulation: solution for injection, Tri-2 Protein Sample, recovered from P. pastoris, 111 mg/mL, in purified Sodium Phosphate 15 mM, Arginine·HCl 100 mM, pH 7.00, 0.2 μm filter
Dose: 10 mg/kg, intramuscular injection (in the neck), starting on D0: approximately 5 h before T0 (challenge) then approximately 3 h post T0. Injections repeated daily, in the morning and approximately 8 h later, from Day 1 (D1) to Day 10 (D10).
   Control Group: Buffer—12 Piglets Challenged on Day 0 at T0 with LT3
VHH: none
Formulation: solution for injection, purified Sodium Phosphate 15 mM, Arginine·HCl 100 mM, pH 7.00, 0.2 μm filter
Dose: same volume as Tri2; intramuscular injection (in the neck), starting on D0: approximately 5 h before T0 (challenge) then approximately 3 h post T0. Injections repeated twice daily, in the morning and approximately 8 h later, from D1 to D10.

### Clinical Observations and Necropsy Observations

Animals observed from D-2 (2 days pre challenge) to D11. Daily measurements of rectal temperature, general health, clinical observations for respiratory signs, local tolerance at injection site and general tolerance.

Body weight at D-7, D-2, D5 and D11 for determination of body weight gain and average daily gain; euthanasia and necropsy at D11 for gross pneumonia lesions scoring (Halbur et al, 1995) and sample collection.

### Sample Collection and Analysis

Blood sample collection at pre-challenge, D2, D5, D7, D9, D11.
   PRRSV qPCR and serology
   Determination of Tri2 concentration.
   Biochemistry analysis to evaluate general tolerance, on pre-challenge and D11 samples only.
Fecal sample collection at the same time points for PRRSV qPCR.
Nasal swabbing for nasal secretions at D11, only for PRRSV qPCR.
Samples collected at necropsy:
   bronchoalveolar fluid for PRRSV qPCR and potentially Tri2 determination; processed for collection of pulmonary alveolar macrophages (PAMs) for potential additional investigations,
   lungs for histopathology to score pneumonia and for PRRSV qPCR,
   tonsils, inguinal lymph node and spleen for PRRSV qPCR.
   Injection sites on both sides of the neck, for histopathology to evaluate local tolerance
   Additional analysis may be conducted on these tissues.

Same sample schedule for the safety group piglets, with additional blood samples collected on D-2 and D-1 to evaluate Tri2 concentration in the hours following administration.

Laboratory examinations, including serum analysis were conducted by personnel masked to treatment allocation.

### Summary of Results

Based on observation of injection sites no abnormalities were reported during the study period indicating that Tri2 was locally well tolerated.

Also, no systemic adverse reactions were observed at any of the clinical observation assessments throughout the study indicating that Tri2 was systemically well tolerated.

Abnormal clinical signs related to challenge were more frequent in the Control group, with sporadic observations of cough in the control group only, and a significantly higher incidence of hyperthermia (rectal temperature ≥40° C.): 45 observations in the control group vs. only 15 in the Tri2 group (P<0.001) (Table 9).

Body weight and body weight gain: groups were consistent and homogenous at study start. At D11 before necropsy, an average difference of approximately 1 kg was observed between the control group (9.7±1.01 kg) and the Tri2 group (10.7±1.67 kg). The average daily gain between D-2 and D11 was significantly higher in the Tri2 group (3.0±0.52 kg) compared to control (2.1±0.71 kg) (p<0.05) (Table 9).

The primary endpoint, PRRSV viral load in serum was significantly lower in Tri2 piglets than in control piglets, at each observation timepoint from D2 to D9 (Table 9). Viral load was assessed by Ct counts, which are inversely proportional to viral load: a Ct of 37 to 40 was considered a negative result and viral load increases when Ct decreases. A difference of approximately 3.3 Ct represents a one log 10 difference in viral titer.

PRRSV serology data is shown in Table 10, suggesting that humoral immune response may develop with a slight delay under Tri2 treatment.

Gross lung lesions, as measured with the Halbur score, were generally less severe in the Tri2 piglets (5.3±5.11) compared to controls (11.1±16.91), but the score difference was not statistically significant probably as a result of data variability and sample size. It is important to note that severely affected piglets, i.e., pigs with a lung lesion score equal to or above 15, were only observed in the control group (4 pigs out of 12. Table 11).

TABLE 9
Summary of clinical and virological observations in pigs
challenged with LT3 and treated with Tri2 or not treated.

| | Treated | Control | Stat. |
|---|---|---|---|
| Parameter | 12 pigs | 12 pigs | analysis |
| RT > 40° C. | 15 observations | 45 observations | P < 0.001 |
| Clinical signs | NA | Sporadic | NA |
| | | Cough in | |
| | | 4 pigs | |
| Daily weight gain | 3.0 ± 0.52 | 2.1 ± 0.71 | P < 0.05 |
| D −2 to D 11 (kg) | | | |
| Gross lung lesions - | 5.26 ± 5.106 | 11.06 ± 16.905 | NS |
| Halbur scoring | | | |
| PCR - Ct count | 40 ± 0.000 | 40 ± 0.000 | NS |
| pre challenge* | | | |
| PCR - Ct count D 2 | 34.72 ± 2.238 | 29.42 ± 2.490 | P < 0.0001 |
| PCR - Ct count D 5 | 31.91 ± 1.843 | 28.06 ± 2.229 | P = 0.0001 |
| PCR - Ct count D 7 | 30.02 ± 1.532 | 26.75 ± 1.514 | P < 0.0001 |
| PCR - Ct count D 9 | 30.04 ± 2.071 | 28.20 ± 1.457 | P = 0.02 |
| PCR - Ct count D 11 | 31.06 ± 2.251 | 30.44 ± 2.470 | NS |

*Ct = 40 considered as a negative result.
Test kit: PCR SDRP EU/M-VBIM/M/008 Biosellal and PCR SDRP US/M-VBIM/M/008 Biosellal

TABLE 10
PRRSV Serology in piglets following
challenge with LT3 strain at D 0

| | D 0 to D 7 | D 9 | D 11 |
|---|---|---|---|
| Treated | all neg | 12 neg | 5 pos/7 neg |
| Control | all neg | 3 pos/9 neg | 10 pos/2 neg |

Neg: negative; pos: positive.
Test kit: IDVET Elisa indirect ID Screen ® PRRS Indirect - IDvet (id-vet.com).

TABLE 11
Distribution of lung lesion scores (scoring
system from Halbur et al, 1995)

| | 0 | >0 to 5 | >5 to 15 | >15 |
|---|---|---|---|---|
| Score | (none) | (mild) | (moderate) | (severe) |
| Treated | 2 | 6 | 4 | 0 |
| Control | 3 | 4 | 1 | 4 |

### CONCLUSION

Treatment of piglets with Tri2 at 10 mg/kg twice daily for 10 days effectively controlled the clinical and virological effects of a PRRSV infection, induced by a PRRSV-1 (LT3) challenge administered after the first Tr2 treatment.

When compared to an untreated control group, Tri2 treatment significantly reduced PRRSV viraemia at all observation timepoints from D2 to D9 post challenge. Seroconversion was observed in both groups with a slight delay in the treated group.

Hyperthermia was significantly less frequent in Tri2 treated piglets. Over a period of 12 days, treated piglets gained an average of 1 kg more than the control piglets and this difference was statistically significant. Gross pneumonia lesions, as observed and scored at necropsy 11 days post challenge were generally milder in the treated group; severe lesions defined as a score >15 were observed in 4 untreated piglets (33%) and none of the treated piglets.

The formulation was well tolerated after multiple intramuscular injections.
Ref: Halbur P G et al. Comparison of the pathogenicity of two US Porcine Reproductive and Respiratory Syndrome Virus isolates with that of the Lelystad virus. Vet. Pathol. 32: 648-660 (1995).

1. An antibody which binds to porcine CD163, wherein said antibody:
(i) binds to the membrane-bound form of porcine CD163 on cells; and
(ii) does not bind significantly to the soluble form of porcine CD163.

2. The antibody of claim 1, wherein said antibody has the ability to bind to the SRCR5 domain of porcine CD163.

3. The antibody of claim 1, or a binding protein, comprising at least one antigen binding domain which binds to porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of GRTFSSYA (SEQ ID NO:2), or a sequence substantially homologous thereto, wherein said substantially homologous sequence is a sequence containing 1, 2 or 3 amino acid substitutions compared to the given CDR sequence,
(ii) a variable heavy (VH) CDR2 that comprises the amino acid sequence of IGWTGGTT (SEQ ID NO:3), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of AADQAGWRTAGVRNTYEYDY (SEQ ID NO:4), or a sequence substantially homologous thereto, wherein said substantially homologous sequence is a sequence containing 1, 2, 3 or 4 amino acid substitutions compared to the given CDR sequence.

4. The antibody or binding protein of claim 3, wherein said heavy chain variable region comprises:
(i) a variable heavy (VH) CDR1 that comprises the amino acid sequence of GRTFSSYA (SEQ ID NO:2),
(ii) a variable heavy (VH) CDR2 that comprises the amino acid sequence of IGWTGGTT (SEQ ID NO:3), and
(iii) a variable heavy (VH) CDR3 that comprises the amino acid sequence of AADQAGWRTAGVRNTYEYDY (SEQ ID NO:4).

5. The antibody of claim 1, comprising a VH domain that comprises the amino acid sequence of SEQ ID NO: 1 or a sequence having at least 70%, 75% or 80% sequence identity thereto.

6. An antibody that binds to the same epitope of porcine CD163 as the antibody of claim 3.

7. A combination of the antibody of claim 1, with one or more further anti-porcine CD163 antibodies or binding proteins, preferably with one or two further anti-porcine CD163 antibodies or binding proteins.

8. The combination of claim 7, wherein each antibody or binding protein binds to a different epitope of porcine CD163.

9. The combination of claim 7, wherein a said further anti-porcine CD163 antibody or binding protein comprises at least one antigen binding domain which binds to porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AISWSGRAPYADSVKG (SEQ ID NO:11), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIKWTTLDAYDY (SEQ ID NO:12), or a sequence substantially homologous thereto, wherein said substantially homologous sequence is a sequence containing 1, 2, 3 or 4 amino acid substitutions compared to the given CDR sequence; and/or
wherein a said further anti-porcine CD163 antibody or binding protein comprises at least one antigen binding domain which binds to porcine CD163, said antigen binding domain comprising a heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:18), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:19), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:20), or a sequence substantially homologous thereto, wherein said substantially homologous sequence is a sequence containing 1, 2, 3 or 4 amino acid substitutions compared to the given CDR sequence.

10. The combination of claim 7, wherein said combination of anti-porcine CD163 antibodies or binding proteins are provided in a single construct, preferably wherein said combination contains two or three anti-porcine CD163 antibodies or binding proteins.

11. The combination of claim 7, wherein one or more, or all, of said antibodies is a single domain antibody.

12. The antibody of claim 1, further comprising an entity that can extend the half-life of said antibody, binding protein or combination, preferably albumin or an IgG Fc region.

13. One or more nucleic acid molecules comprising nucleotide sequences that encode the antibody of claim 1, or one or more expression vectors comprising such nucleic acid molecules, or one or more host cells comprising said expression vectors or said nucleic acid molecules or expressing the antibody of claim 1.

14. (canceled)

15. (canceled)

16. A method of producing an antibody, said method comprising the steps of (i) culturing a host cell of claim 13, under conditions suitable for the expression of the encoded antibody; and optionally (ii) isolating or obtaining the antibody from the host cell or from the culture medium/supernatant.

17. A composition comprising an antibody of claim 1, one or more nucleic acid molecules encoding said antibody, one or more expression vectors comprising said nucleic acid molecules, or one or more host cells comprising said expression vectors, or said nucleic acid molecules, or expressing the antibody of claim 1.

18. (canceled)

19. (canceled)

20. A method of treatment or prevention of PRRSV infection in a subject, which method comprises administering to a subject in need thereof a therapeutically effective amount of the combination of claim 7, one or more nucleic acid molecules encoding said combination, one or more expression vectors comprising said nucleic acid molecules, or one or more host cells comprising said expression vectors, or said nucleic acid molecules, or expressing the combination of claim 7.

21. (canceled)

22. The method of claim 20, wherein said subject is a pig.

23. A method of treatment or prevention of PRRSV infection in a subject, which method comprises administering to a subject in need thereof a therapeutically effective amount of the antibody of claim 1, one or more nucleic acid molecules encoding said antibody, one or more expression vectors encoding said nucleic acid molecules, or one or more host cells comprising said expression vectors, or said nucleic acid molecules, or expressing the antibody of claim 1.

24. The method of claim 23, wherein said subject is a pig.

25. The antibody of claim 1, wherein said antibody is a single domain antibody.


SEQUENCE LISTING (44 sequences)

SEQ ID NO: 1 - protein, 127 aa, synthetic construct
QVQLVESGGGLVQAGGSLRLSCAASGRTFSSYAMGWFRQTPGKDREFVARIGWTGGTTIYADSVKGRFTISRDNAKNTVYLQMNSLKPEDTAVYYCAADQAGWRTAGVRNTYEYDYWGQGTQVTVSS

SEQ ID NO: 2 - protein, 8 aa, synthetic construct
GRTFSSYA

SEQ ID NO: 3 - protein, 8 aa, synthetic construct
IGWTGGTT

SEQ ID NO: 4 - protein, 20 aa, synthetic construct
AADQAGWRTAGVRNTYEYDY

SEQ ID NO: 5 - protein, 25 aa, synthetic construct
QVQLVESGGGLVQAGGSLRLSCAAS

SEQ ID NO: 6 - protein, 17 aa, synthetic construct
MGWFRQTPGKDREFVAR

SEQ ID NO: 7 - protein, 38 aa, synthetic construct
IYADSVKGRFTISRDNAKNTVYLQMNSLKPEDTAVYYC

SEQ ID NO: 8 - protein, 11 aa, synthetic construct
WGQGTQVTVSS

SEQ ID NO: 9 - protein, 123 aa, synthetic construct
QVQLQESGGGLVQVGSSLRLSCVTSGRTPSRYVMGWFRQAPGQEREFVAAISWSGRAPYADSVKGRFTISRDNAKNTVYLQMNSLKPEDTGVYYCAGGEGAIKWTTLDAYDYWGQGTQVTVSS

SEQ ID NO: 10 - protein, 5 aa, synthetic construct
RYVMG

SEQ ID NO: 11 - protein, 16 aa, synthetic construct
AISWSGRAPYADSVKG

SEQ ID NO: 12 - protein, 15 aa, synthetic construct
GEGAIKWTTLDAYDY

SEQ ID NO: 13 - protein, 30 aa, synthetic construct
QVQLQESGGGLVQVGSSLRLSCVTSGRTPS

SEQ ID NO: 14 - protein, 14 aa, synthetic construct
WFRQAPGQEREFVA

SEQ ID NO: 15 - protein, 32 aa, synthetic construct
RFTISRDNAKNTVYLQMNSLKPEDTGVYYCAG

SEQ ID NO: 16 - protein, 11 aa, synthetic construct
WGQGTQVTVSS

SEQ ID NO: 17 - protein, 129 aa, synthetic construct
QLQLVESGGGLVQPGGSLRLSCAASGFTLDDYTIGWFRQAPGKEREGVSCINSITSNTYYADSVKGRFTISRDNAKNTVYLQMNSLTAEDTAIYYCAADSGLFSGSSCLKYRAMRFGSWGQGTQVTVSS

SEQ ID NO: 18 - protein, 5 aa, synthetic construct
DYTIG

SEQ ID NO: 19 - protein, 17 aa, synthetic construct
CINSITSNTYYADSVKG

SEQ ID NO: 20 - protein, 20 aa, synthetic construct
DSGLFSGSSCLKYRAMRFGS

SEQ ID NO: 21 - protein, 30 aa, synthetic construct
QLQLVESGGGLVQPGGSLRLSCAASGFTLD

SEQ ID NO: 22 - protein, 14 aa, synthetic construct
WFRQAPGKEREGVS

SEQ ID NO: 23 - protein, 32 aa, synthetic construct
RFTISRDNAKNTVYLQMNSLTAEDTAIYYCAA

SEQ ID NO: 24 - protein, 11 aa, synthetic construct
WGQGTQVTVSS

SEQ ID NO: 25 - protein, 123 aa, synthetic construct; identical to SEQ ID NO: 9

SEQ ID NO: 26 - protein, 8 aa, synthetic construct
GRTPSRYV

SEQ ID NO: 27 - protein, 7 aa, synthetic construct
ISWSGRA

SEQ ID NO: 28 - protein, 17 aa, synthetic construct
AGGEGAIKWTTLDAYDY

SEQ ID NO: 29 - protein, 25 aa, synthetic construct
QVQLQESGGGLVQVGSSLRLSCVTS

SEQ ID NO: 30 - protein, 17 aa, synthetic construct
MGWFRQAPGQEREFVAA

SEQ ID NO: 31 - protein, 38 aa, synthetic construct
PYADSVKGRFTISRDNAKNTVYLQMNSLKPEDTGVYYC

SEQ ID NO: 32 - protein, 11 aa, synthetic construct
WGQGTQVTVSS

SEQ ID NO: 33 - protein, 129 aa, synthetic construct; identical to SEQ ID NO: 17

SEQ ID NO: 34 - protein, 8 aa, synthetic construct
GFTLDDYT

SEQ ID NO: 35 - protein, 8 aa, synthetic construct
INSITSNT

SEQ ID NO: 36 - protein, 22 aa, synthetic construct
AADSGLFSGSSCLKYRAMRFGS

SEQ ID NO: 37 - protein, 25 aa, synthetic construct
QLQLVESGGGLVQPGGSLRLSCAAS

SEQ ID NO: 38 - protein, 17 aa, synthetic construct
IGWFRQAPGKEREGVSC

SEQ ID NO: 39 - protein, 38 aa, synthetic construct
YYADSVKGRFTISRDNAKNTVYLQMNSLTAEDTAIYYC

SEQ ID NO: 40 - protein, 11 aa, synthetic construct
WGQGTQVTVSS

SEQ ID NO: 41 - protein, 101 aa, Sus scrofa
PRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCS

SEQ ID NO: 42 - protein, 1115 aa, Sus scrofa
MDKLRMVLHENSGSADFRRCSAHLSSFTFAVVAVLSACLVTSSLGGKDKELRLTGGENKCSGRVEVKVQEEWGTVCNNGWDMDVVSVVCRQLGCPTAIKATGWANFSAGSGRIWMDHVSCRGNESALWDCKHDGWGKHNCTHQQDAGVTCSDGSDLEMGLVNGGNRCLGRIEVKFQGRWGTVCDDNFNINHASVVCKQLECGSAVSFSGSANFGEGSGPIWFDDLVCNGNESALWNCKHEGWGKHNCDHAEDAGVICLNGADLKLRVVDGVTECSGRLEVKFQGEWGTICDDGWDSDDAAVACKQLGCPTAVTAIGRVNASEGTGHIWLDSVSCHGHESALWQCRHHEWGKHYCNHDEDAGVTCSDGSDLELRLKGGGSHCAGTVEVEIQKLVGKVCDRSWGLKEADVVCRQLGCGSALKTSYQVYSKTKATNTWLFVSSCNGNETSLWDCKNWQWGGLSCDHYDEAKITCSAHRKPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCSRYTQIRLVNGKTPCEGRVELNILGSWGSLCNSHWDMEDAHVLCQQLKCGVALSIPGGAPFGKGSEQVWRHMFHCTGTEKHMGDCSVTALGASLCSSGQVASVICSGNQSQTLSPCNSSSSDPSSSIISEENGVACIGSGQLRLVDGGGRCAGRVEVYHEGSWGTICDDSWDLNDAHVVCKQLSCGWAINATGSAHFGEGTGPIWLDEINCNGKESHIWQCHSHGWGRHNCRHKEDAGVICSEFMSLRLISENSRETCAGRLEVFYNGAWGSVGRNSMSPATVGVVCRQLGCADRGDISPASSDKTVSRHMWVDNVQCPKGPDTLWQCPSSPWKKRLASPSEETWITCANKIRLQEGNTNCSGRVEIWYGGSWGTVCDDSWDLEDAQVVCRQLGCGSALEAGKEAAFGQGTGPIWLNEVKCKGNETSLWDCPARSWGHSDCGHKEDAAVTCSEIAKSRESLHATGRSSFVALAIFGVILLACLIAFLIWTQKRRQRQRLSVFSGGENSVHQIQYREMNSCLKADETDMLNPSGDHSEVQ

SEQ ID NO: 43 - protein, 5 aa, synthetic construct
GGGGS

SEQ ID NO: 44 - protein, 10 aa, synthetic construct
GGGGSGGGGS